(19) United States
(12) Patent Application Publication
(10) Pub. No.: US 2009/0092991 A1
(43) Pub. Date: Apr. 9, 2009

(54) Assays, methods and systems for predicting follicular lymphoma outcome

(76) Inventors: Harriet E. Feilotter, Gananoque (CA); David P. LeBrun, Kingston (CA); Tara D. Baetz, Kingston (CA); Karen J. Harrison, Kingston (CA); Roland Somogyi, Sydenham (CA); Larry D. Greller, Kingston (CA)

Correspondence Address: Licata & Tyrrell P.C., 66 E. Main Street, Marlton, NJ, 08053, US

(21) Appl. No.: 12/283,659

(22) Filed: Sep. 15, 2008

Related U.S. Application Data
(60) Provisional application No. 60/972,056, filed on Sep. 13, 2007.

(51) Int. Cl. C12Q 1/68; G01N 33/48

(52) U.S. Cl. 435/6; 702/20

(57) ABSTRACT

Assays, kits, methods and systems for predicting outcome in patients with follicular lymphoma based upon measurement of one or more phenomenologically competitive or synergistic gene pairs or a set of classifier genes are provided.

This patent application claims the benefit of priority from U.S. Provisional Application Ser. No. 60/972,056, filed Sep. 13, 2007, teachings of which are incorporated herein in their entirety.

### FIELD OF THE INVENTION

The present invention provides an assay or kit for predicting outcome in patients with follicular lymphoma based upon measurement and/or evaluation of expression levels of one or more gene pairs or a selected set of classifier genes, levels of which are predictive of 5 year survival in the patient. The present invention also provides methods and computer systems for prognosticating outcome of a patient with follicular lymphoma based upon measurement and/or evaluation of expression levels of one or more of these gene pairs or a selected set of classifier genes.

### BACKGROUND OF THE INVENTION

Non-Hodgkin's lymphoma (NHL) is the fifth most frequent cancer in North America. Follicular lymphoma (FL) is the second most prevalent NHL lymphoma type, responsible for 24-40% of lymphoma cases (Naresh et al. Leuk. Lymphoma 2004 45:1569-1577; Naresh et al. Blood 1997 89:3909-3918). FL is generally considered an indolent lymphoma; most patients experience prolonged survival, initially with little or no specific therapy (Lopez-Guillermo et al. Leuk. Lymphoma 1994 15: 159-165; Solal-Celigny et al. Blood 2004 104:1258-1265). However, some cases pursue a more aggressive clinical course and realize relatively short survival.

Current strategies to stratify FL cases into clinically relevant subtypes, including histological grading and application of clinical parameters such as those used to compute the Follicular Lymphoma International Prognostic Index (FLIPI), offer only modest prognostic capability and clinical utility (Perea et al. Ann. Oncol. 2005 September; 16(9):1508-13. Epub 2005 Jun. 6). Candidate biomarkers of outcome in patients with FL, include bcl2 (Ott et al. Blood 2002 99:3806-3812; Hoglund et al. Genes Chromosomes Cancer 2004 39:195-204; Buchonnet et al. Leukemia 2002 16:1852-1856; Menendez et al. Leukemia 2004 18:491-498; Noriega et al. Blood Cells Mol. Dis. 2004 32:232-239; Buchonnet et al. Leukemia 2000 14:1563-1569; Cleary et al. J. Exp. Med. 1986 164:315-320; Lestou et al. Br. J. Haematol 2003 122:745-759; Fenton et al. 2002 Blood 99:716-718; Mandigers et al. Ann. Hematol. 2003 82:743-749; Gascoyne et al. Blood 1997 90:244-251}, p53 (Ott et al. Blood 2002 99:3806-3812; Martinez-Climent et al. Blood 2003 101:3109-3117; Sander et al. Blood 1993 82:1994-2004; Lossos et al. Semin. Cancer Biol. 2003 13:191-202) and myc (Lossos et al. Proc. Natl. Acad. Sci. USA 2002 99:8886-8891). However, none of these candidates have been shown to be markedly superior to the clinical indices already available.

More recently, results from global gene expression profiling studies using primary tumor samples have uncovered alterations in specific signal transduction pathways and contributions from non-neoplastic cells in the tumor microenvironment that correlate with clinical parameters (Dales et al. Mol. Pathol. 2001 54:17-23; Robetorye et al. J. Mol. Diagn. 2002 4:123-136; Elenitoba-Johnson et al. Proc. Natl. Acad. Sci. USA 2003 100:7259-7264; Dave et al. N Engl J Med 2004 351(21):2159-2169; Glas et al. Blood 2005 105:301-307; Goy et al. Cancer 2006 108:10-20; Luminari, S, and Federico, M. Hematol. Oncol. 2006 24:64-72; Hui et al. Mol. Pathol. 2006 19:1192-1202). Such studies have identified common themes with respect to the genes whose expression levels differentiate outcomes in FL. Pathways that have been implicated include apoptosis, cell cycling, T-cell markers, and signaling pathways that involve c-myc.

For example, the role of apoptosis in the development of FL is documented, with multiple studies suggesting that down-regulation of pro-apoptotic and up-regulation of anti-apoptotic genes are associated with poor outcome in FL (Naresh et al. Blood 1997 89:3909-3918; Dales et al. Mol. Pathol. 2001 54:17-23; Elenitoba-Johnson et al. Proc. Natl. Acad. Sci. USA 2003 100:7259-7264; Hui et al. Mol. Pathol. 2006 19:1192-1202; Paterson et al. Haematologica 2006 91:772-780; Lossos et al. Proc. Natl. Acad. Sci. USA 2002 99:8886-8891).

Overexpression of NOTCH2 has also been reported in a number of human lymphoma cell lines (Jundt et al. Blood 2002 103:3511-3515; Kapp et al. J. Exp. Med. 1999 189:1939-1946), and appears to be involved in increased cell survival and proliferation (Troen et al. J. Mol. Diagn. 2004 6:297-307). In mouse, NOTCH2 activity has been shown to be required for proper B cell development, suggesting a role in cellular proliferation and differentiation (Saito et al. Immunity 2003 18:675-685; Witt et al. 2003 Mol. Cell. Biol. 23:8637-8650).

In addition, TFF3 overexpression has been implicated in increasing invasiveness and decreasing apoptosis in a number of human cell lines (Emami et al. Peptides 2001 25:885-898; Rodrigues et al. Faseb J. 2001 15:1517-1528).

Further, expression of PLA2G3, part of the ERK/MAPK signaling pathway, has been demonstrated to reliably distinguish diffuse large B cell lymphoma (DLBCL) from FL cases (Elenitoba-Johnson et al. Proc. Natl. Acad. Sci. USA 2003 100:7259-7264), and appears to have a role in stimulating both tumor cell growth (Han et al. J. Biol. Chem. 2004 279:44344-44354) and angiogenesis (Murakami et al. 2005 J. Biol. Chem. 280:24987-24998), and protecting against apoptosis (Casas et al. J. Biol. Chem. 2006 281:6106-6116).

A microarray study of FL has demonstrated that cells from the tumor microenvironment may be driving the gene expression patterns linked to outcome. In that study, an immune response 1 signature was associated with good outcome, while the immune response 2 signature was associated with poor outcome. It was hypothesized that the expression patterns in the good outcome signature were derived from T-cells and monocytes, while that in the poor outcome signature was derived from monocytes and dendritic cells (Dave et al. N. Engl. J. Med. 2004 351:2159-2169).

However, care must be taken when comparing results between these studies, since different experimental approaches have been taken and subtly different questions have been asked. For instance, some studies have used cell lines (Robetorye et al. J. Mol. Diagn. 2002 4:123-136), while others have used material from primary tumors (Martinez-Climent et al. Blood 2003 101:3109-3117; Lossos et al. Proc. Natl. Acad. Sci. USA 2002 99:8886-8891; Elenitoba-Johnson et al. Proc. Natl. Acad. Sci. USA 2003 100:7259-7264; Dave et al. N. Engl. J. Med. 2004 351:2159-2169; Glas et al. Blood 2005 105:301-307; Hui et al. Mol. Pathol. 2006 19:1192-1202; Bohen et al. Proc. Natl. Acad. Sci. USA 2003 100:1926-1930). Some studies have investigated the effects of treatment on outcome (Bohen et al. Proc. Natl. Acad. Sci. USA 2003 100:1926-1930; Harjunpaa et al. Br. J. Haematol. 2006 135:33-42) while others have ensured that the samples are derived from untreated tumors (Dave et al. N. Engl. J. Med. 2004 351:2159-2169). Some studies have microdissected tumor cells for study (Husson et al. Blood 2002 99:282-289), while others have investigated the tumor in its microenvironment (Dave et al. N. Engl. J. Med. 2004 351:2159-2169). Additionally, many of the microarray studies have compared matched samples of pre- and post-transformation FL (Lossos et al. Proc. Natl. Acad. Sci. USA 2002 99:8886-8891; Elenitoba-Johnson et al. Proc. Natl. Acad. Sci. USA 2003 100:7259-7264).

New molecular profile-based prognostic tests derived more directly from observed human patient biology, morbidity, and disease are needed for follicular lymphoma.

### SUMMARY OF THE INVENTION

An aspect of the present invention relates to an assay or kit for predicting outcome in patients with follicular lymphoma based upon detection and/or evaluation of expression levels of one or more gene pairs or a selected set of classifier genes predictive of 5 year survival in the patient. Preferably gene expression levels are determined in a tumor sample. In one embodiment, the assay or kit detects expression levels of one or more gene pairs of Table 2. In another embodiment, the assay or kits detects expression levels of a selected set of classifier genes.

Another aspect of the present invention relates to a method for prognosticating outcome in patients with follicular lymphoma, said method comprising detecting and/or evaluating levels of expression of one or more gene pairs or a selected set of classifier genes predictive of 5 year survival in the patient. In one embodiment, expression levels of one or more gene pairs of Table 2 are detected and/or evaluated. In another embodiment, expression levels of a selected set of classifier genes are detected and/or evaluated.

Another aspect of the present invention relates to a computer system predictive of the outcome of patients with follicular lymphoma. The computer system comprises a central processing unit, a memory connected to the central processing unit, said memory storing established levels of expression of one or more gene pairs or a selected set of classifier genes predictive of 5 year survival or of death within 5 years derived from individuals with follicular lymphoma, and/or multiple gene expression levels of a patient, a computer program capable of comparing levels of expression of said predictive gene pairs or said selected set of classier genes in the patient with stored levels, and instructions for outputting predicted outcome of the patient.

### BRIEF DESCRIPTION OF FIGURES

FIG. 1 provides expression intensity matrix (samples vs. genes) plots illustrating various single gene performance of the top 20 ranking up-regulated genes and the top 20 down-regulated genes that distinguish 5 year survival outcomes according to t-test p-values on differences of class means. For comparison, outcome prediction accuracies, as represented by sensitivity, positive predictive value (PPV), specificity, and negative predictive value (NPV) are reported, where sensitivity=TPs/(TPs+FNs), specificity TNs/(TNs+FPs), PPV (Positive Predictive Value)=TPs/(TPs+FPs), NPV (Negative Predictive Value)=TNs/(TNs+FNs), and where TPs are # true positive samples, TNs are # true negative samples, FPs are # false positive samples, and FNs are # false negative samples. Average and standard deviations of the performance values of these 40 select genes are also shown at the bottom of the columns. The black and white numbering scheme emphasizes the expression level with respect to each profile's average and standard deviation (s.d.), i.e., 3 and 4 signify positive deviation from the mean (0 s.d. to <1 s.d. 3, >=1 s.d. 4) and 1 and 2 signify negative deviation from the mean (0 s.d. to >−1 s.d. 2, <=−1 s.d. 1), as shown in the plot legend.

FIGS. 2a and 2b provides expression intensity matrix (samples vs. gene pairs) plots illustrating gene pair performances. FIG. 2a shows intensity profiles of replicated gene pairs found independently as combinations of microarray features with strong outcome discriminating p-values. FIG. 2b shows the top 40 ranking gene pairs according to t-test p-values. In addition to outcome prediction gene pair p-values, outcome prediction accuracies, as represented by sensitivity, PPV, specificity and NPV are also reported. Average and standard deviations of the performance values of these 40 select gene pairs are also shown at the bottom of the columns. The numbering scheme emphasizes the intensity levels with respect to each profile's average and standard deviation (s.d.) as shown in the numbering scheme legends of the figures.

FIGS. 3a and 3b provide detailed Predictive Interaction Analysis (PIA) model performance characterization for the top-performing competitive gene pair example NOTCH2-RIPK5. FIG. 3a shows measurement values, means, and standard deviation bars for each of the four PIA model variables, i.e., single genes x, y and gene pairs u, v. Gene x corresponds to NOTCH2 (single gene p=10-2.7) and gene y corresponds to RIPK5 (single gene p=10^{-3.6}). The best-performing model (competitive) clearly stands out in terms of increased separation of means relative to shrinking standard deviations as v (top panel of 4 panels) (gene pair p=10^{-7.4}). FIG. 3b shows detailed two-dimensional (2d) scatterplots of PIA models for the same top-performing competitive gene pair example. The 2d visualization illustrates the diagonal position of the PIA separatrix with slope +1 for the competitive model, in comparison to the one-dimensional (1d) model separatrices (shown as vertical and horizontal broken lines). This emphasizes how the PIA diagonal separatrix improves outcome class separation compared to the single gene x and single gene y respective vertical and horizontal separatrices. Specifically, for the competitive v model sensitivity=88% (2 5yd misclassifications) and specificity=80%, (5 5ya misclassifications) are observed (where 5yd denotes death less than 5 years after diagnosis and 5ya means alive 5 years after diagnosis), compared to the constituent gene x, NOTCH2, with sensitivity=88% (2 5yd misclassifications) and specificity=72%, (7 5ya misclassifications), and gene y, RIPK5, with sensitivity=75% (4 5yd misclassifications) and specificity=72%, (7 5ya misclassifications).

FIGS. 4a and 4b show cross-validation robustness to added zero-mean Gaussian simulated noise for best performing PIA model. Simulated noise was added numerically to the original measurement values for each of the 5000 training/test set splits used in the cross-validation. The average cross-validation accuracy is plotted as a function of amplitude of added simulated noise in standard deviation units, using the standard deviation of the original measurements. FIG. 4a shows results for sensitivity and FIG. 4b shows results for specificity. The PIA model example is for the synergistic gene pair LOXL3 and NTS, gene pair p=10^{-8.0.}

FIGS. 5a and 5b show results for overall survival of patients grouped by combinatorial gene pair classification. Kaplan-Meier analysis of overall survival for 41 patients grouped according to outcome classification was performed based on the LOXL3 and NTS gene pair in FIG. 5a and the RIPK5 and NOTCH2 gene pair in FIG. 5b. Log-rank statistical test was used to assess the difference of two survival curves.

FIGS. 6a, 6b and 6c show results of overall survival of patients grouped by FLIPI risk category. Kaplan-Meier analysis of overall survival comparing pooled low and intermediate FLIPI risk versus high FLIPI risk was performed and is depicted in FIG. 6a (n=41); Kaplan-Meier analysis of overall survival comparing LOXL3 and NTS PIA based gene pair outcome classification in combined FLIPI low risk and intermediate risk patients is depicted in FIG. 6b (n=18). Kaplan-Meier analysis of overall survival of FLIPI high risk patients is depicted in FIG. 6c. The log-rank statistical test was used to assess differences of two survival curves

FIG. 7 shows results from a single classifier for a correct-class partition of a primary dataset consisting of expression data for 41,000 genes from 29 tissue samples of follicular lymphoma classified as having “good” (alive 5 years after diagnosis) or “poor” (dead within three years of diagnosis) outcome with tumors having a DLBCL component excluded. The two axes represent different weighted averages of the genes involved. Poor outcomes are x's (training) and squares (test) and good outcomes are +'s (training) and *'s (test).

FIG. 8 shows a single classifier for a random-class partition. The two axes represent different weighted averages of the genes involved. Poor outcomes are x's (training) and squares (test) and good outcomes are +'s (training) and *'s (test). The test and training data are not notably correlated.

FIG. 9 shows classification based on Average 1. Classification accuracy is 85% for poor-outcome samples (11/13) and 87% for good-outcome samples (14/16). Good samples are +'s, poor are x's. Weights are negative, so the lower “good” values have a weighted average higher than the “poor” values, although the “poor” expression levels are in fact elevated over the “good” expression levels (also see FIG. 10.)

FIG. 10 shows the mean and standard deviation for genes from a 13-gene set of classifier genes. Error bars show 1 standard deviation.

### DETAILED DESCRIPTION OF THE INVENTION

The present invention relates to expression-based, gene-pair assays, kits, methods and computer systems for predicting poor FL outcomes. The inventors herein have now found that certain genes in statistically conservative gene pair models and selected sets of classifier genes exhibit high predictive accuracy when using death within a specified period of time from diagnosis as an endpoint in patients with FL.

Gene pairs and sets of classifier genes to be detected and/or evaluated in the assays and methods of the present invention were identified by examination of non-dissected material from primary tumors. The endpoint was defined using the outcome measure of death within 5 years of diagnosis, as this endpoint is biologically relevant yet simple and easy to determine. The biomarkers predictive of clinical outcome in FL described herein were identified by performing gene expression profiling on primary lymphoma biopsy samples.

Using PIA (Predictive Interaction Analysis; Baron et al. PLOS Med. 2007 4:e23), gene pairs were identified whose expression patterns are capable of predicting death within 5 years of diagnosis in this dataset with ANOVA p-values below 10⁻⁷, and outcome prediction accuracies exceeding 85%, a performance that is far superior to that achieved by the FLIPI score on the same dataset.

PIA is a recently reported fundamental computational method for identifying synergistic and competitive phenomenological relationships from measured pair-wise levels of activity variables (genes) in distinguishing key biomedical outcomes or phenotypes (Baron et al. PLOS Med. 2007 4:e23). PIA is conservative statistically, easily computed, and has minimal data requirements. It also is readily applicable to complex and voluminous studies. PIA is inherently numerically data-driven and free of literature-based biases or subjectivity regarding gene interactions.

In the PIA method, gene pairs are represented by the constructed single variables v=x−y for so-called “competitive” PIA models, and u=x+y for so-called “synergistic” PIA models, x and y being the log₁₀ expression levels of each gene of any given pair. “Phenomenologically competitive” means good prediction of outcome using v=x −y, i.e., X/Y in the original unlogged measurements. “Phenomenologically synergistic” means good prediction of outcome using u=x+y, i.e., X*Y in the original unlogged measurements. The single gene x and y, and gene pair v and u variables, were analyzed for their abilities to discriminate death within 5 years after diagnosis from death beyond 5 years after diagnosis using the conventional 2-tailed, heteroscedastic Student's t-test for difference of two means. Two quantitative criteria were defined for measuring outcome-prediction interaction effects. These were: 1) “Stringent p-value gain”, measured for comparison of gene pair performance to the best constituent single gene performance, and 2) “principal p-value gain”, measured for comparison of gene pair performance to the null model which assumes that gene pair expression was not correlated within each class. Only gene pairs with both stringent and principal p-value gains ≧10 times that of the best of their respective independent genes' models were considered for further prioritization and analysis.

PIA requires a minimum of two biological sample classes (e.g., death before 5 years vs. alive beyond 5 years). It requires at least two continuous ordinal activity variables (e.g., expression levels of two genes) to define a phenomenological interaction. The two simplest forms of interactions are termed synergistic and competitive predictive interaction analysis, abbreviated SPIA and CPIA, respectively. While interaction of any activity variables associated with a biological outcome can be analyzed using this method, for the present invention we focused on gene expression data, and refer to the expression level of a gene simply as “gene”. Synergistic or competitive interactions were then defined respectively as the product or quotient of levels of two genes.

Empirically, gene expression abundances are generally log-normal-like distributed across genes; hence, we usually worked with log-transformed abundances to obtain more bell-shaped Gaussian-like distributions to better meet the Gaussian distributional assumptions common to most gene expression statistical analyses. The detailed description and specification of PIA follows:

u=log(x*y)=log(x)+log(y), and v=log(x/y)=log(x)−log(y) for the respective SPIA and CPIA models.

From here on, x will then refer to log(x), and y will then refer to log(y). SG (single gene) x and SG (single gene) y are described as the constituent variables, and u and v are described as the derived respective synergistic and competitive GP (gene pair) variables.

In PIA, classical statistical distributional methods are then used to determine the associations of x, y, u, v with the two class outcomes and to provide class-separation performance scores. Specifically, the heteroscedastic Student's t-test provides p-values to assess separation of the means of class-specific distributions against the null hypothesis of no difference between class means. LDA (linear discriminant analysis) provides the model for predicting the outcome class of a GP.

For convenience, “abslogp” is defined as the absolute value of the log₁₀ of a statistical p-value. Two-class separation can be assessed using an abslogp obtained from a t-test under the no separation null hypothesis. For a given GP and its constituent SGs, abslogp values can be obtained using x only, y only, u only, and v only. The GP u or v model abslogp (whichever of the two is stronger) is then compared to the best SG model abslogp. The “stringent abslogp gain” is defined to establish whether the SPIA or CPIA GP model performs better than the constituent SGs in class discrimination:

stringent abslogp gain=max (u abslogp, v abslogp)−max (x abslogp, y abslogp).

Stringent abslogp gain is a good measure for defining GP performance as compared to best constituent SG performance, strictly in terms of outcome discrimination.

However, it does not provide a complete enough characterization statistically of the phenomenological interaction strength of the two constituent SGs. This is because only the stronger one of the constituent SGs comes into this measure rather than both SGs simultaneously.

Principal abslogp remedies this lack of completeness in a principled, though subtle, way. “Principal abslogp gain” compares observed GP performance to expected GP performance in a null model that assumes that for each class there is no within-class interaction between the constituent SG variables. This distributional PIA GP null model is constructed by using the observed distributional properties of the SG variables, and then determining the GP t-test performance based on the assumption that there is no interaction between the SGs, i.e., that the (x,y)-variables constituting the u and v variables are uncorrelated within each class.

The null model is implemented through manipulation of the variance of u and the variance of v as follows. A theoretical or observed within-class standard deviation in the u-model is always given in terms of x and y standard deviations as σᵤ=\sqrt{σₓ²+σ_{y}²=2ρ(x,y)σₓσ_{y}}, where ρ(x,y) is Pearson correlation between x and y. This is because always var(u)=var(x)+var(y)+2 cov(x,y). Analogously, σᵥβ\sqrt{σₓ²+σ_{y}²−2ρ(x,y)σₓσ_{y}} because always var(v)=var(x)+var(y)−2cov(x,y) (Taylor J. R., An Introduction to Error Analysis, University Science Books, Mill Valley, California, 1982; P K MacKeown, Stochastic Simulation in Physics, Springer-Verlag, Singapore, 1997). The above formulae for u and v standard deviations are special cases of the long-established general so-called “propagation of error” technique (Eisenhart, C. and Zelin, M. Ch. 12 Elements of Probability, E. U. Condon & H. Odishaw, eds., Handbook of Physics, McGraw-Hill, N.Y., 1958, p. 1-143).

Thus in SPIA, two-class separation t-test abslogp is computed using the actual data (which inherently has within-class standard deviations given by σᵤ). The cognate null model t-test abslogp is then computed using a σ₇=\sqrt{σₓ²+σ_{y}²} where ρ(x,y) is required to be explicitly zero rather than what it actually is. The difference between the class-separation abslogp's so-computed is the SPIA Principal abslogp. The analogous calculations are performed for CPIA Principal abslogp.

Prediction outcome related accuracies were then calculated. For all single gene and gene pair predictive models, 4 types of LDA (linear discriminant analysis) outcome class prediction accuracies are reported. The regions predictive of the positive event (death within 5 years, “poor outcome”) and negative event (alive after 5 years, “good outcome”) outcomes were separated by the point equidistant to the means of both outcome groups, i.e., the linear discriminant analysis point-separatrix. The counts of true positive (TP), true negative (TN), false positive (FP) and false negative (FN) samples were then determined according to whether a sample was correctly or incorrectly classified for each class. Classification accuracies were defined as follows: Sensitivity=TPs/(TPs+FNs), Specificity=TNs/(TNs+FPs), PPV (Positive Predictive Value)=TPs/(TPs+FPs), NPV (Negative Predictive Value)=TNs/(TNs+FNs).

The gene pairs identified for detection and/or evaluation in the assay of the present invention are pairs that are predictive of 5 year survival in the patient. Without being limited to a specific mechanism of action and/or theory, these gene pairs are believed to be phenomenologically competitive or synergistic based upon their identification via PIA. Examples of PIA software developed in accordance with teachings of Baron et al. (PLOS Med. 2007 4:e23) include, but are not limited to, PIA implementation, cross-validations, and simulated noise additions of MATLAB™ program codes using the MATLAB™, versions 6.x-7.x, programming language and the MATLAB™ Statistics Toolbox, versions 4.x-5.x, (MATLAB™ programming language products available from The MathWorks, Inc.(Natick Mass.)). These phenomenologically competitive or synergistic gene pairs are believed to be associated with the cell survival pathways. For example, using the Ingenuity Pathways Analysis (IPA) software tool (Ingenuity® Systems, Redwood City, Calif., USA) genes from the gene pair predictor list were grouped into canonical pathway including apoptosis (NOTCH2, TFF3, CSF1, BNIP1, BCLAF1, BMX, BIRC4, SRF), chemokine signaling (LIMK1, ROCK2, CCL13), cell growth and proliferation (BIRC4, BMX, CSF1, DGKA, TNFRSF6B, CSF1, LIMK1, SRF), and hematological function (CCL13, CD47, CSF1, TNFRSF6B, GREM1, PLA2G4A, NOTCH2, GATA3, MMRN¹, TSPAN8). Several pro-apoptotic genes are among the most consistently down-regulated genes, including BCLAF1, BIRC4 (also XIAP) and RIPK5, while the up-regulated genes include anti-apoptotic genes NOTCH2, TFF3, CSF1 and BMX. Thus, preferred phenomenologically competitive or synergistic gene pairs detected and/or evaluated in the assays and methods of the present invention are pairs selected from genes involved in apoptosis, for example, but not limited to, NOTCH2, TFF3, CSF1, BNIP1, BCLAF1, BMX, BIRC4 and SRF, genes involved in chemokine signaling, for example, but not limited to, LIMK1, ROCK2, and CCL13, genes involved in cell growth and proliferation, for example, but not limited to, BIRC4, BMX, CSF1, DGKA, TNFRSF6B, CSF1, LIMKl and SRF, and genes involved in hematological function As shown herein, these gene pairs exhibit both higher sensitivity and specificity than single genes in predicting FL outcome. For example, for the 40 gene pairs with the best 2-tailed heteroscedastic t-test p-values for difference of outcome class mean expression, also referred to herein as the “top 40” or “top” gene pairs and depicted herein in Table 2, an average sensitivity of 87% and an average specificity of 79% were observed, compared to the averages of the top 40 single genes of 78% and 65%, respectively. A preferred synergistic gene pair model for measurement in an assay of the present invention is based on the PIA-distinguished outcome class-based phenomenological (competitive or synergistic) interaction of LOXL3 and NTS and exhibits a sensitivity of 94%, and specificity of 84%. Measurement and/or evaluation of expression levels of one or more of the phenomenologically competitive or synergistic genes pairs identified herein provides a prognostic tool for application in clinical settings to assist clinicians in the development of individualized patient care for those suffering from FL.

Expression levels of single gene members that constitute one or more of the gene pairs or a set of classifier genes predictive of 5 year survival in the patient can be measured and/or evaluated by various assays. By measurement of gene expression levels as used herein it is meant to be inclusive of measurement of RNA molecules isolated from a patient such as messenger RNA as well as fragments thereof. In some cases, protein levels expressed by one or more of the gene pairs are measured as discussed hereinbelow.

Gene expression levels may be measured by any method known in the art including, but not limited to, measuring mRNA expression by Northern blot, quantitative reverse transcriptase PCR(RT-PCR) via, for example, Taqman® (Applied Biosystems, Foster City, Calif.) or QuantiTect® SYBR systems (Qiagen, Valencia, Calif.), Molecular Beacons® (Public Health Research Institute, Newark, N.J.) which uses a probe having a fluorescent molecule and being capable of hairpin structure formation and a quencher molecule, microarray, dot or slot blots, in situ hybridization or SAGE (serial amplification of gene expression). See, e.g., Sambrook et al., Molecular Cloning: A Laboratory Manual, 2d ed., Cold Spring Harbor Laboratory Press (1989); Sambrook et al., Molecular Cloning: A Laboratory Manual, 3d ed., Cold Spring Harbor Press (2001); Ausubel et al., Current Protocols in Molecular Biology, Greene Publishing Associates (1992, and Supplements to 2000); and Ausubel et al., Short Protocols in Molecular Biology: A Compendium of Methods from Current Protocols in Molecular Biology—4^{th} Ed., Wiley & Sons (1999).

By evaluating gene expression levels, as used herein, it is meant that the measured gene expression levels in a patient are analyzed, examined and/or compared to determine if the levels at which gene pair(s) or a set of classifier genes predictive of outcome in follicular lymphoma are expressed and, if expressed, if such gene pair expression levels or expression levels of the set of classifier genes are indicative of poor outcome or good outcome in the patient.

The present invention provides kits for conducting such assays. Components provided in the kits of the present invention will depend upon the assay format.

For example, a kit for measuring and/or evaluating gene expression levels via quantitative RT-PCR comprises primer pairs specific for a gene pair or a set of classifier genes predictive of 5 year survival in the patient identified herein. Such kits may comprise multiple primer pairs, each primer pair being specific for a different gene of the gene pairs identified herein. In general, the primers are at least 10 nucleotides in length, more preferably at least 12, more preferably at least 14 and even more preferably at least 16 or 17 nucleotides in length and are derived from a gene of a gene pair or a set of classifier genes predictive of 5 year survival in the patient identified herein. These kits may also comprise dNTPs and/or Taq polymerase as well as a probe or probes specific for the gene pair or pairs or set of classifier genes and/or an intercalating dye such as QuantiTect™M SYBR® Green.

Alternative methods of performing primer-directed amplification are also well known in the art. Methods for performing the polymerase chain reaction (PCR) are compiled, inter alia, in McPherson, PCR Basics: From Background to Bench, Springer Verlag (2000); Innis et al. (eds.), PCR Applications: Protocols for Functional Genomics, Academic Press (1999); Gelfand et al. (eds.), PCR Strategies, Academic Press (1998); Newton et al., PCR, Springer-Verlag New York (1997); Burke (ed.), PCR: Essential Techniques, John Wiley & Son Ltd (1996); White (ed.), PCR Cloning Protocols: From Molecular Cloning to Genetic Engineering, Vol. 67, Humana Press (1996); and McPherson et al. (eds.), PCR 2: A Practical Approach, Oxford University Press, Inc. (1995). Methods for performing RT-PCR are collected, e.g., in Siebert et al. (eds.), Gene Cloning and Analysis by RT-PCR, Eaton Publishing Company/Bio Techniques Books Division, 1998; and Siebert (ed.), PCR Technique: RT-PCR, Eaton Publishing Company/BioTechniques Books (1995).

Isothermal amplification approaches, such as rolling circle amplification, are also well-described. See, e.g., Schweitzer et al., Curr. Opin. Biotechnol. 2001 12(1): 21-7 and U.S. Pat. Nos. 5,854,033 and 5,714,320. Rolling circle amplification can be combined with other techniques to facilitate gene expression level detection. See, e.g., Lizardi et al. Nature Genet. 1998 19(3): 225-32.

Kits for measuring gene expression levels via a hybridization assay are also provided. In one embodiment, such kits comprise at least two probes, each probe being derived from a gene of a gene pair predictive of 5 year survival in the patient identified herein. In another embodiment the kit comprises a probe derived from one or more genes in a set of classifier genes identified herein predictive of 5 year survival in the patient. In general, the probes are oligonucleotides at least 10 nucleotides in length, more preferably at least 12, more preferably at least 14 and even more preferably at least 16 or 17 nucleotides in length. Methods of performing nucleic acid hybridization using oligonucleotide probes are well known in the art. See, e.g., Sambrook et al., 1989, supra, Chapter 11 and pp. 11.31-11.32 and 11.40-11.44, which describes radiolabeling of short probes, and pp. 11.45-11.53, which describes hybridization conditions for oligonucleotide probes, including specific conditions for probe hybridization (pp. 11.50-11.51). Such kits may further comprise means for detecting the probes.

Alternatively, if a microarray approach is used wherein expression levels of multiple gene pairs or a set of classifier genes identified herein are determined, the kit comprises a nucleic acid microarray having a substrate-bound plurality of nucleic acids, hybridization to each of the plurality of bound nucleic acids being separately detectable. The substrate can be solid or porous, planar or non-planar, unitary or distributed. Exemplary nucleic acid microarrays include, but are not limited to devices such as described in Schena (ed.), DNA Microarrays: A Practical-Approach (Practical Approach Series), Oxford University Press (1999); Nature Genet. 21(1)(suppl.):1-60 (1999); Schena (ed.), Microarray Biochip Tools and Technology, Eaton Publishing Company/BioTechniques Books Division (2000). Additionally, these nucleic acid microarrays include a substrate-bound plurality of nucleic acids in which the plurality of nucleic acids are disposed on a plurality of beads, rather than on a unitary planar substrate, as is described, inter alia, in Brenner et al. Proc. Natl. Acad. Sci. USA 2000 97(4):1665-1670. Examples of nucleic acid microarrays may be found in U.S. Pat. Nos. 6,391,623, 6,383,754, 6,383,749, 6,380,377, 6,379,897, 6,376,191, 6,372,431, 6,351,712 6,344,316, 6,316,193, 6,312,906, 6,309,828, 6,309,824, 6,306,643, 6,300,063, 6,287,850, 6,284,497, 6,284,465, 6,280,954, 6,262,216, 6,251,601, 6,245,518, 6,263,287, 6,251,601, 6,238,866, 6,228,575, 6,214,587, 6,203,989, 6,171,797, 6,103,474, 6,083,726, 6,054,274, 6,040,138, 6,083,726, 6,004,755, 6,001,309, 5,958,342, 5,952,180, 5,936,731, 5,843,655, 5,814,454, 5,837,196, 5,436,327, 5,412,087, 5,405,783. Preferably the nucleic acid microarray detects expression levels of 30-50 different genes of the gene pairs identified herein.

It is expected that assays and kits capable of detecting and/or evaluating levels of proteins expressed by one or more of the gene pairs or a set of classifier genes identified as predictive of 5 year survival in the patient can also be used to predict poor FL outcome. Protein levels expressed by one or more of the gene pairs or the set of classifier genes may be determined by any method known in the art including, but not limited to, radioimmunoassays, competitive-binding assays, ELISA, Western blot, FACS, immunohistochemistry, immunoprecipitation, proteomic approaches: two-dimensional gel electrophoresis (2D electrophoresis) and non-gel-based approaches such as mass spectrometry or protein interaction profiling. Accordingly, kits for conducting such methods may comprise antibodies specific to selected proteins expressed by of one or more of the gene pairs or the set of classifier genes and/or means for detecting bound antibodies. Alternatively, the kits may comprise a peptide microarray or a protein microarray having a substrate-bound collection of plurality of polypeptides, the binding to each of the plurality of bound polypeptides being separately detectable, or a plurality of binders, including but not limited to monoclonal antibodies, polyclonal antibodies, phage display binders, yeast 2 hybrid binders and aptamers, which can specifically detect the binding of the expressed proteins of gene pairs or the set of classifier genes of this invention. Exemplary peptide microarrays are set forth in U.S. Pat. Nos. 6,268,210, 5,766,960, 5,143,854.

Kits of the present invention may further comprise one or more controls. Examples of a positive control for use in the present invention include, but are not limited to: a microarray or chip with a control well(s) comprising a gene pair or a set of classifier genes predictive of poor (or good) FL outcome at levels associated with poor (or good) FL outcome; a microarray or chip with control wells, each control well comprising a gene of a gene pair or set of classifier genes predictive of poor (or good) FL outcome at expression levels associated with poor (or good) FL outcome, and table or chart of established levels of expression of one or more gene pairs or set of classifier genes predictive of 5 year survival or death within 5 years of individuals with follicular lymphoma. Examples of a negative control for use in the present invention are genes and/or gene pairs not associated with follicular lymphoma, expression levels of which are measured to establish a baseline brightness (signal) level of the microarray or chip.

The above kits of the present invention can be used with any RNA or protein containing biological sample from a patient. Examples of such biological samples include, but are not limited to, tumor biopsies, blood, plasma, serum, white blood cells, lymph and urine.

The following embodiments are described with respect to gene pairs indicative of poor outcome in a patient. The invention also encompasses corresponding embodiments of gene pairs indicative of good outcome in a patient as well as embodiments with respect to a set of classifier genes indicative of poor or good outcome in a patient.

In one embodiment, a microarray or microchip assay and/or kit of the present invention is used as follows. RNA isolated from a tumor sample from a patient is contacted with the microarray or microchip. The expression brightness (i.e., intensity) of each of the single genes from the patient's chip is determined. All the gene pairs from the chip and the respective expression brightnesses of the single genes that constitute each gene pair are then determined. Over all the gene pairs from the patient's chip, the chip's average and standard deviation expression brightness are then calculated in the u model and in the v model. Then, for a specific gene pair on the patient's chip, its expression brightness (in the u or v model) is compared as being at least a certain number of brightness standard deviations different from the chip's mean brightness. A difference in brightness of 3-fold, more preferably 5-fold, more preferably 10-fold, and even more preferably 100-fold is indicative of differential expression of the gene pair and poor outcome for the patient.

Alternatively, the expression brightness (in the u or v model) of a certain gene pair measured in the patient can be compared to the mean brightness of a set of calibrant/control gene pairs that are on the patient's chip. In this embodiment, a difference in the number of brightness standard deviations from the control of, for example, 3-fold, more preferably 5-fold, more preferably 10-fold, and even more preferably 100-fold could be indicative of differential expression of the gene pair and poor outcome for the patient. Alternatively, the control gene pair on the chip may be at a level indicative of differential expression of the gene pair and poor outcome for the patient. In this embodiment, a similar brightness in the sample from the patient would be indicative of poor outcome for the patient.

In yet another embodiment, a 3-threshold rule for outcome-prediction callout can be used. In this embodiment, a gene pair predictive of poor FL outcome, referred to herein for exemplary purposes as #k, is represented or encoded with 3 threshold criteria (xk, yk, zk). Expression brightness is then measured for the patient and evaluated as follows: if the expression brightness of the 1st gene of the gene pair is greater than xk; and the expression brightness of the 2nd gene of the gene pair is greater than yk; and the sum (e.g., for a u-model gene pair) of the two single gene brightnesses is greater than zk, then the brightness measured in the patient for this gene pair predicts that the patient is likely to die within 5 years of diagnosis. In this embodiment, the respective (1st gene, 2nd gene, gene pair) threshold values (xk, yk, zk) can be in brightness units that are determined for the entire chip or can be defined in terms of a set of calibrant/control single genes and gene pairs average and standard deviations on the chip.

To identify gene pairs predictive of poor FL outcome, 23,216 features were analyzed across 41 tumor tissue samples from patients with FL. Key baseline data collected on all patients and samples in the study are summarized in Table 1.

TABLE 1
Patient Characteristics

| | 41 Patient Set |
|---|---|
| Patient Characteristic | n(%)* |
| Sex | |
| Female | 23 (56%) |
| Male | 18 (44%) |
| Age | |
| ≦60 years | 21 (51%) |
| >60 years | 20 (49%) |
| Ann Arbor Stage | |
| I or II | 15 (38%) |
| III or IV | 25 (63%) |
| Hemoglobin Level | |
| <120 g/L | 13 (35%) |
| ≧120 g/L | 24 (65%) |
| Number of Nodal Areas | |
| ≦4 | 22 (56%) |
| >4 | 17 (44%) |
| Serum Lactate Dehydrogenase | |
| Normal | 25 (86%) |
| Elevated | 4 (14%) |
| ECOG Performance Status | |
| ≦1 | 33 (85%) |
| >1 | 6 (15%) |
| Number of Extranodal Sites | |
| ≦1 | 38 (97%) |
| >1 | 1 (3%) |
| Tumour Grade^{†} | |
| 1 | 19 (49%) |
| 2 | 6 (15%) |
| 3 | 14 (36%) |
| Median Follow-Up Time^{†} | |
| All Patients | 6.1 years |
| Patients Still Alive | 7.2 years |

*Percentage of the patients for which we have data for that characteristic.
^{†}All other variables are baseline clinical characteristics of the patient at diagnosis.

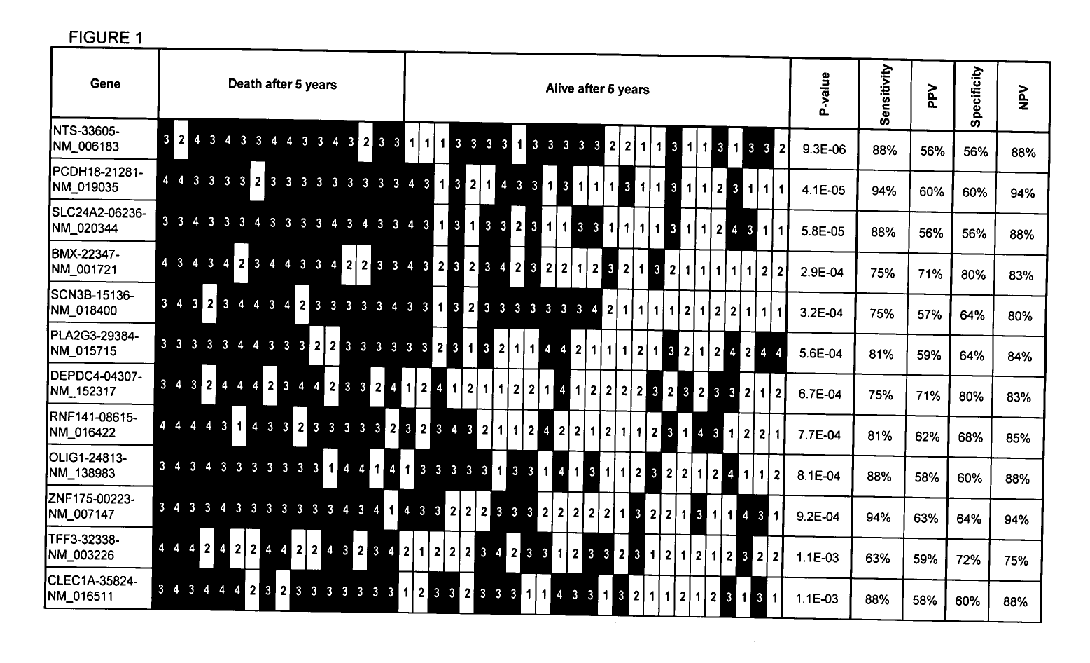

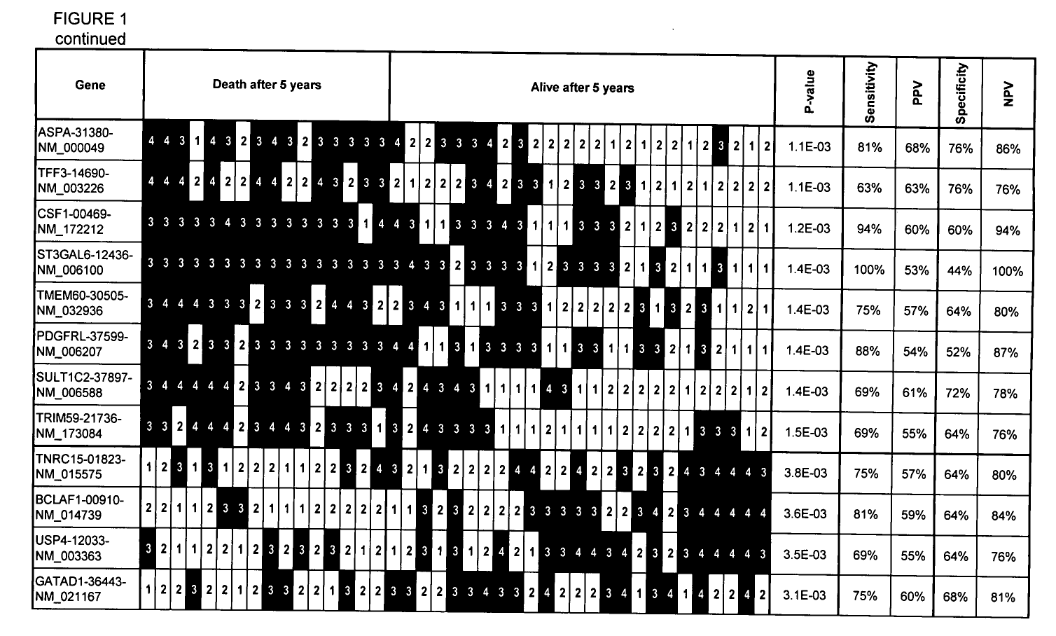

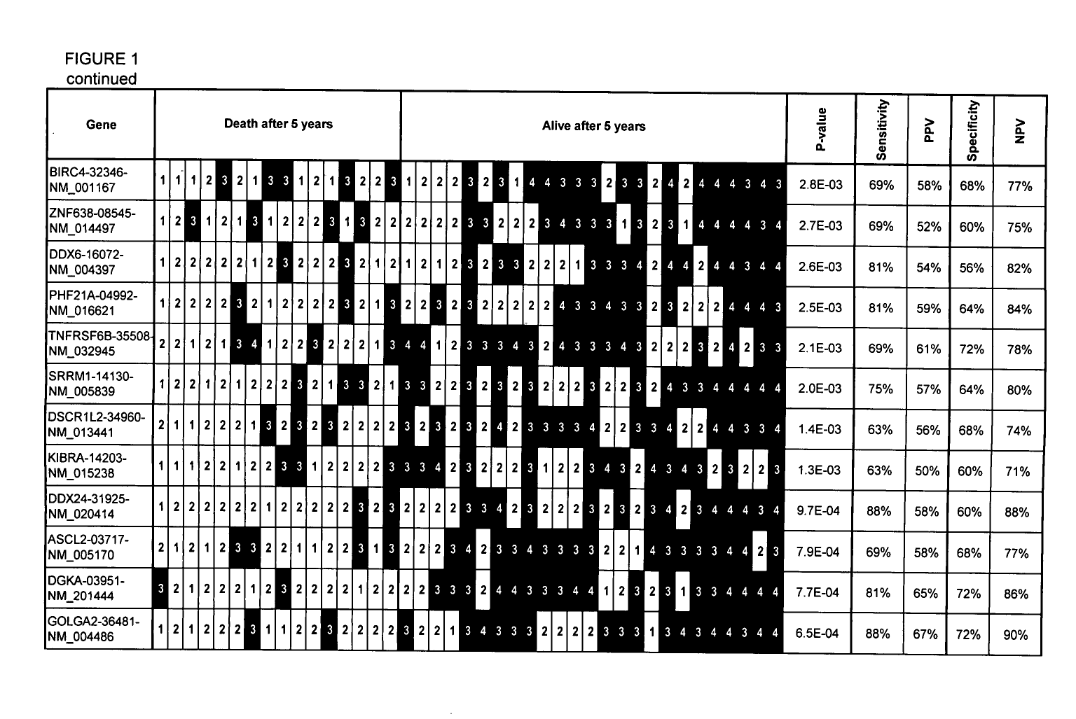

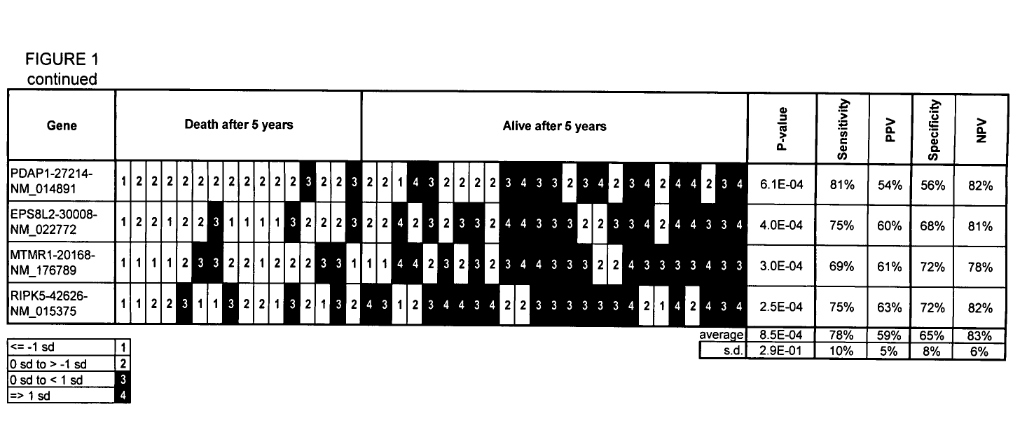

For each single gene, a 2-tailed (i.e., 2-sided) heteroscedastic Student's t-test (null hypothesis: no difference between mean of class 1 samples and mean of class 2 samples) for discriminating death within 5 years of diagnosis (i.e., class 1) from death after 5 years from diagnosis (i.e., class 2) was carried out on all 23,216 features (expression intensity values measured from the 41 tumors). Single genes were than ranked in order from strongest (i.e., smallest) to weakest (i.e., largest) t-test p-value for rejection of the no difference between class means null hypothesis. The top 300 best performing single genes from this ranking with respect to p-value were selected. The numerical values of p-values associated with the predictive capabilities, i.e., the discrimination of the two classes, i.e., t-test p-values under the no difference of class means null hypothesis, of these 300 top genes ranged from 9.3×10⁻⁶ to 0.013. These 300 top single genes served as the input for PIA. PIA then analyzes each possible pair of genes from the top 300 single genes, i.e., 300*299/2=44,850 gene pairs. Because neither a pre-selected nor data-derived threshold-for-significance p-value is involved when using this gene-ranking-by-p-value approach, there is no need to adjust the numerical significance values of the t-test p-values for multiple hypothesis tests being employed, namely, that many thousands of single genes were tested. The top 20 up-regulated and down-regulated gene expression profiles are shown in FIG. 1.

Seven genes in this list, namely ST3GAL6, PLA2G4A, BMX, CASP4, DGKA, TFF3 and SRRM1, were represented by more than one feature on the array, and were discovered independently with strong p-values and highly replicated expression patterns. The genes ST3GAL6 and PLA2G4A are represented by a single probe sequence each, and are each spotted in 10 different locations over the chip. Eight of the ten ST3GAL6 and seven of the ten PLA2G4A features were selected as having similar predictive value in the data set. Several other genes are represented by either two (BMX, CASP4, DGKA) or three (TFF3, SRRMl) different probe sequences. In this analysis, both probes representing BMX, CASP4 and DGKA, and two of three probes representing TFF3 and SRRM1 were identified as having similar predictive value.

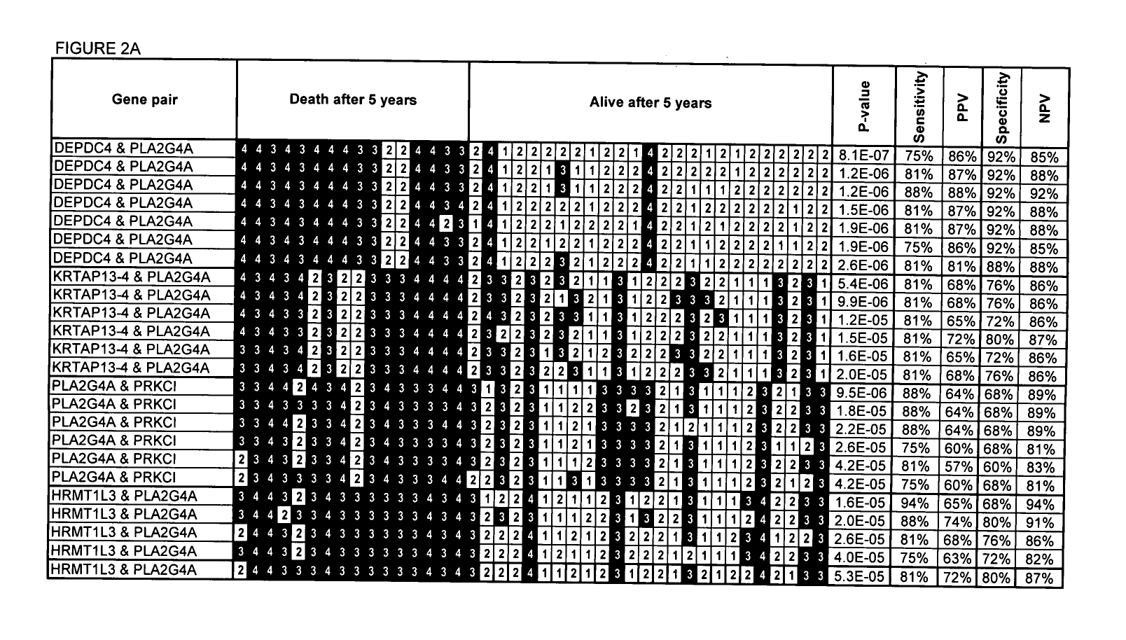

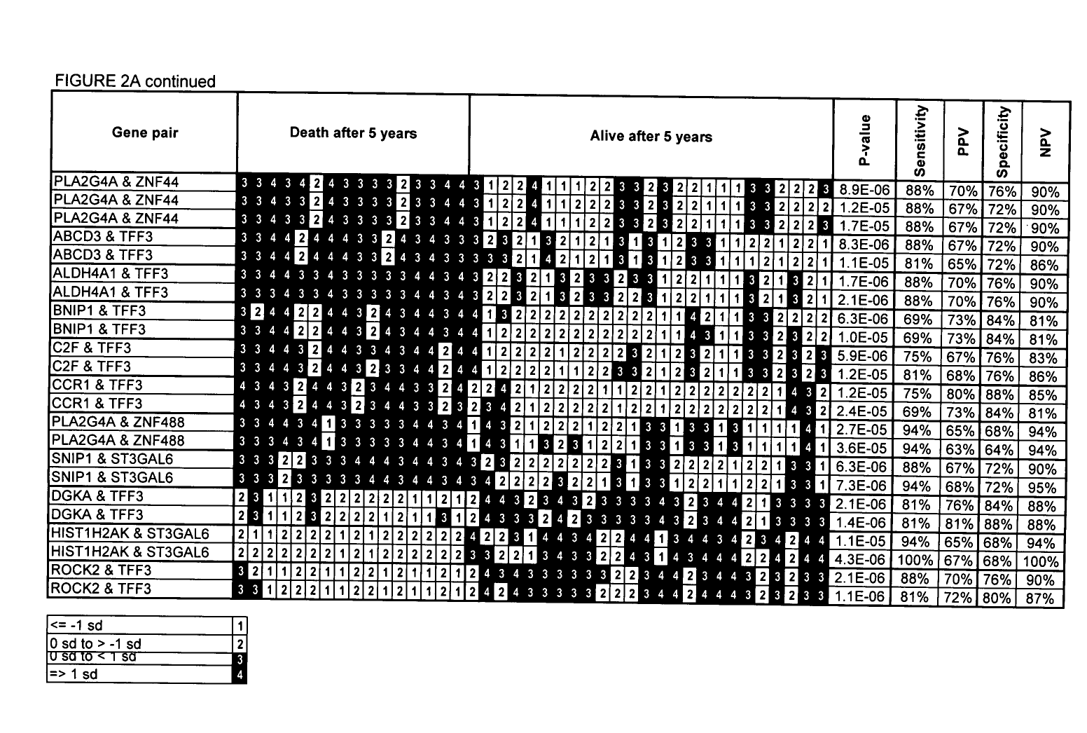

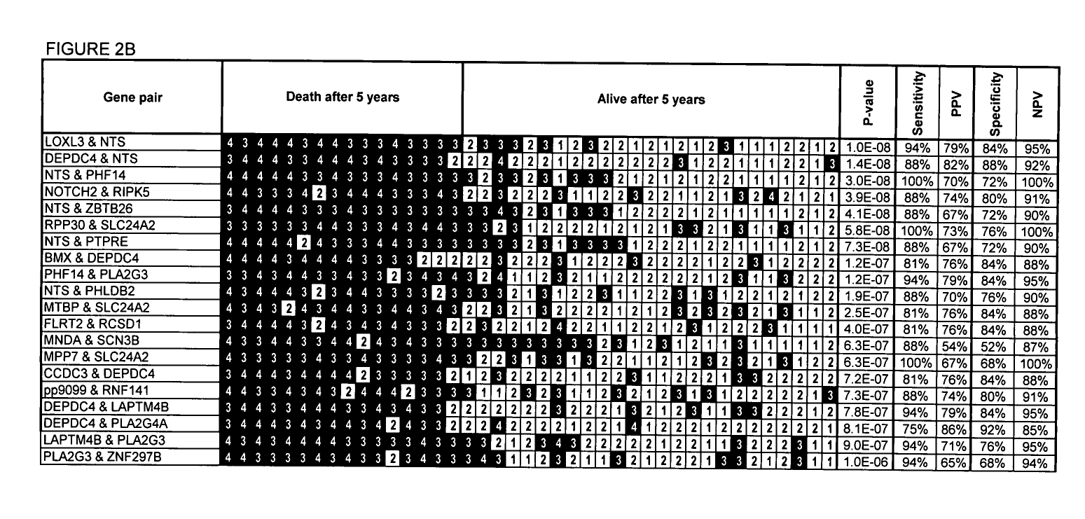

PIA was carried out to examine whether any of the 44,850 gene pairs generated from the 300 single genes were able to discriminate the 5-year outcomes more reliably than either single gene of the pair. To be conservative, only gene pairs with both stringent and principal p-value gains of >=10 times (when written as p-values, not log(p-values)) that of their respective independent gene models were considered for further prioritization and analysis. The principle of this analytical approach was that FL outcomes are too complex to be predicted by just one gene, but that a good predictive model is built with a minimum number of genes using statistically conservative methods. The employed implementation of PIA limits gene interactions to pairs that are either phenomenologically competitive or synergistic. These are encoded simply as ratios (differences of logs) or products (sums of logs), respectively, of the constituent single gene variables, essentially generating a single variable representing each gene pair. The variables are subjected to statistical t-tests, to identify those that best distinguish the “event does not occur” or “negative” event outcome class (alive throughout 5 years) from the “event occurs” or “positive” event outcome class (death within 5 years). Using this approach, it is clear that the best gene pairs outperform the best single genes in class-discriminating p-values by approximately 3 orders of magnitude (see FIGS. 1 and 2). Moreover, for the four different outcome discriminating accuracy measures disclosed herein, an approximately 10% improvement in gene pairs compared to single genes was seen (see FIGS. 1 and 2).

Of the 303 gene pairs that passed both of the phenomenological gene-interaction performance criteria, 15 repeated gene pairs were observed due to redundant features or genes represented by multiple probes on the array (see FIG. 2a). P-values and predictive accuracies were averaged for these redundant gene pairs.

Overall, 271 non-redundant gene pairs (comprising 178 constituent single genes) remained, with p-value gains ranging from 104 to 108 for discriminating 5 year outcomes. The top-performing gene pairs according to two-class discrimination 2-tailed heteroscedastic t-test p-value performance are shown in FIG. 2b. The best performing gene pair models showed 1000-fold lower p-values than the best single genes shown in FIG. 1. For both single and pair gene models, it is generally observed that sensitivity and negative predictive value (NPV) perform better than specificity and positive predictive value (PPV). The models performed very well, sometimes perfectly, in correctly identifying the positive (death within 5 years) and negative (alive after 5 years) events. However, this is sometimes accompanied by a weakness in PPV and specificity, i.e., some of the samples classified as positive are truly negative (PPV), and concomitantly, not all of the negative samples have been correctly found (specificity). That is, not all gene pairs are good predictors of prognosis since some are weaker in specificity and/or selectivity. Preferred gene pairs for detection and/or evaluation in accordance with the present invention are set forth in Table 2.

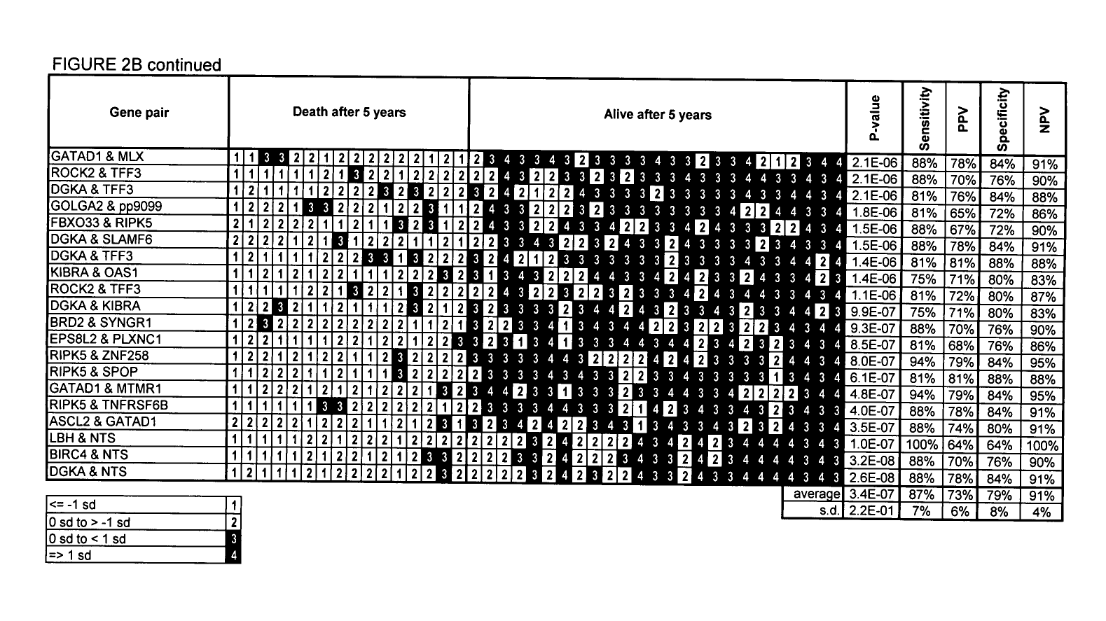

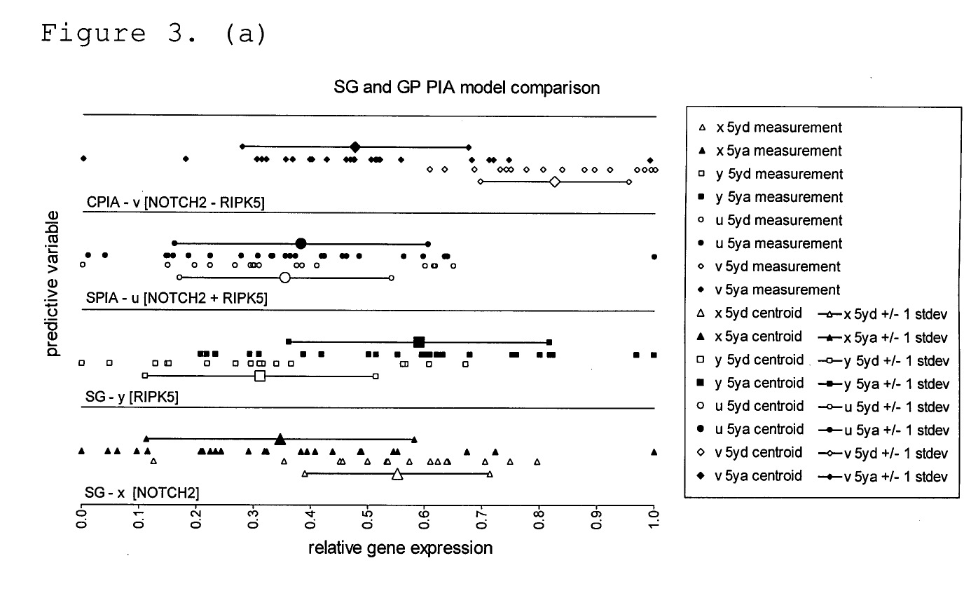

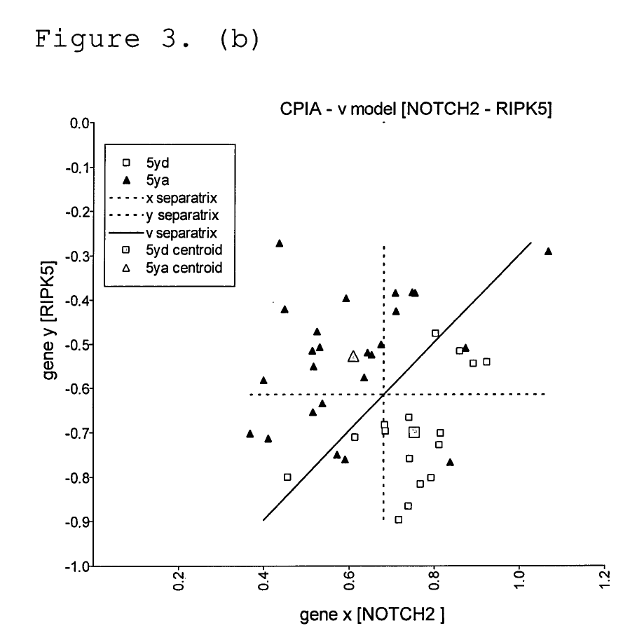

Plots for the best performing competitive gene pair in terms of 5 year outcome prediction performance, NOTCH2-RIPK5, are presented in one and two-dimensional visualizations in FIG. 3. The one dimensional graphs in FIG. 3a display the measurement points and key statistical features of the class-discriminating t-test, i.e., the mean and standard deviations for each class. By visually comparing the results from the synergistic and competitive gene pair and the y and x single gene variables (SPIA-u, CPIA-v, SG-y and SG-x, respectively, it clearly can be seen that the competitive model provides at once the largest separation of means, and least overlap of the standard deviations, which explains the superior v variable p-value of 3.9*10⁻⁸ (top panel of 4 panels). The full two-dimensional display of the measurement data in FIG. 3b illustrates how much better the PIA separatrix (solid diagonal line) performs in separating the classes, compared to the single gene separatrices (horizontal and vertical broken lines). Similar interaction strengths were observed with the best synergistic gene pairs.

The PIA-derived gene pairs capture reproducible and statistically supported competitive and synergistic interactions between genes, providing t-test p-values for each predictive pair. Table 2 contains the 5000 data splits cross-validation averages, standard deviations, and coefficients of variation for sensitivity, specificity, PPV, and NPV for the top 40 gene pairs. In this table, death within 5 years is referred to as “positive” event (i.e., a death “event” observed within 5 years) and alive throughout 5 years as “negative” event (i.e., no death “event” observed within 5 years). Cross-validation was carried out on 5000 different selections of training/test dataset splits, by randomly selecting 75% of the samples for training (12 positive and 19 negative samples), and 25% of the samples for testing (4 positive and 6 negative samples). Table 2 shows the average accuracy values over all 5000 training/test dataset splits for sensitivity [TP/(TP+FN)], specificity [TN/(TN+FP)], PPV [TP/(TP+FP)], and NPV [TN/(TN+FN)], and their standard deviations and coefficients of variation (TP=true positive, TN=true negative, FP=false positive, FN=false negative).

TABLE 2

| | | Cross- | Cross- | Cross- | | Cross- | Cross- | Cross- | | Cross- |
|---|---|---|---|---|---|---|---|---|---|---|
| | Full | validation | validation | validation | Full | validation | validation | validation | Full | validation |
| | dataset | sensitivity | sensitivity | sensitivity | dataset | PPV | PPV | PPV | dataset | specificity |
| Gene pair | sensitivity | average | s.d. | coeff. var. | PPV | average | s.d. | coeff. var. | specificity | average |
| LOXL3 & | 0.94 | 0.91 | 0.15 | 0.16 | 0.79 | 0.81 | 0.14 | 0.18 | 0.84 | 0.83 |
| NTS | | | | | | | | | | |
| DEPDC4 & | 0.88 | 0.85 | 0.17 | 0.20 | 0.82 | 0.87 | 0.14 | 0.16 | 0.88 | 0.89 |
| NTS | | | | | | | | | | |
| NTS & | 1.00 | 0.96 | 0.10 | 0.11 | 0.70 | 0.72 | 0.13 | 0.18 | 0.72 | 0.72 |
| PHF14 | | | | | | | | | | |
| NOTCH2 & | 0.88 | 0.88 | 0.15 | 0.17 | 0.74 | 0.77 | 0.15 | 0.19 | 0.80 | 0.80 |
| RIPK5 | | | | | | | | | | |
| NTS & | 0.88 | 0.89 | 0.15 | 0.17 | 0.67 | 0.71 | 0.15 | 0.21 | 0.72 | 0.73 |
| ZBTB26 | | | | | | | | | | |
| RPP30 & | 1.00 | 0.97 | 0.08 | 0.08 | 0.73 | 0.78 | 0.15 | 0.19 | 0.76 | 0.78 |
| SLC24A2 | | | | | | | | | | |
| NTS & | 0.88 | 0.85 | 0.18 | 0.22 | 0.67 | 0.72 | 0.17 | 0.23 | 0.72 | 0.74 |
| PTPRE | | | | | | | | | | |
| BMX & | 0.81 | 0.78 | 0.20 | 0.26 | 0.76 | 0.81 | 0.16 | 0.20 | 0.84 | 0.86 |
| DEPDC4 | | | | | | | | | | |
| PHF14 & | 0.94 | 0.92 | 0.13 | 0.14 | 0.79 | 0.81 | 0.14 | 0.18 | 0.84 | 0.83 |
| PLA2G3 | | | | | | | | | | |
| NTS & | 0.88 | 0.86 | 0.16 | 0.19 | 0.70 | 0.73 | 0.15 | 0.21 | 0.76 | 0.76 |
| PHLDB2 | | | | | | | | | | |
| MTBP & | 0.81 | 0.85 | 0.18 | 0.21 | 0.76 | 0.81 | 0.16 | 0.20 | 0.84 | 0.84 |
| SLC24A2 | | | | | | | | | | |
| FLRT2 & | 0.81 | 0.80 | 0.20 | 0.26 | 0.76 | 0.79 | 0.16 | 0.20 | 0.84 | 0.84 |
| RCSD1 | | | | | | | | | | |
| MNDA & | 0.88 | 0.88 | 0.15 | 0.17 | 0.54 | 0.59 | 0.14 | 0.23 | 0.52 | 0.55 |
| SCN3B | | | | | | | | | | |
| MPP7 & | 1.00 | 1.00 | 0.01 | 0.01 | 0.67 | 0.70 | 0.13 | 0.18 | 0.68 | 0.68 |
| SLC24A2 | | | | | | | | | | |
| CCDC3 & | 0.81 | 0.79 | 0.21 | 0.27 | 0.76 | 0.80 | 0.16 | 0.20 | 0.84 | 0.85 |
| DEPDC4 | | | | | | | | | | |
| pp9099 & | 0.88 | 0.86 | 0.17 | 0.19 | 0.74 | 0.77 | 0.16 | 0.20 | 0.80 | 0.80 |
| RNF141 | | | | | | | | | | |
| DEPDC4 & | 0.94 | 0.90 | 0.15 | 0.16 | 0.79 | 0.80 | 0.14 | 0.18 | 0.84 | 0.83 |
| LAPTM4B | | | | | | | | | | |
| DEPDC4 & | 0.75 | 0.76 | 0.20 | 0.26 | 0.86 | 0.88 | 0.15 | 0.16 | 0.92 | 0.92 |
| PLA2G4A | | | | | | | | | | |
| LAPTM4B & | 0.94 | 0.88 | 0.17 | 0.20 | 0.71 | 0.72 | 0.15 | 0.21 | 0.76 | 0.74 |
| PLA2G3 | | | | | | | | | | |
| PLA2G3 & | 0.94 | 0.90 | 0.16 | 0.18 | 0.65 | 0.69 | 0.14 | 0.21 | 0.68 | 0.69 |
| ZNF297B | | | | | | | | | | |
| GATAD1 & | 0.88 | 0.82 | 0.20 | 0.24 | 0.78 | 0.79 | 0.16 | 0.21 | 0.84 | 0.82 |
| MLX | | | | | | | | | | |
| ROCK2 & | 0.88 | 0.82 | 0.22 | 0.26 | 0.70 | 0.72 | 0.16 | 0.22 | 0.76 | 0.76 |
| TFF3 | | | | | | | | | | |
| DGKA & | 0.81 | 0.78 | 0.20 | 0.25 | 0.76 | 0.80 | 0.17 | 0.21 | 0.84 | 0.85 |
| TFF3 | | | | | | | | | | |
| GOLGA2 & | 0.81 | 0.82 | 0.18 | 0.22 | 0.65 | 0.69 | 0.16 | 0.23 | 0.72 | 0.73 |
| pp9099 | | | | | | | | | | |
| FBXO33 & | 0.88 | 0.87 | 0.16 | 0.18 | 0.67 | 0.70 | 0.15 | 0.22 | 0.72 | 0.71 |
| RIPK5 | | | | | | | | | | |
| DGKA & | 0.88 | 0.88 | 0.15 | 0.17 | 0.78 | 0.81 | 0.16 | 0.19 | 0.84 | 0.84 |
| SLAMF6 | | | | | | | | | | |
| DGKA & | 0.81 | 0.81 | 0.18 | 0.23 | 0.81 | 0.83 | 0.16 | 0.20 | 0.88 | 0.86 |
| TFF3 | | | | | | | | | | |
| KIBRA & | 0.75 | 0.76 | 0.20 | 0.26 | 0.71 | 0.72 | 0.18 | 0.25 | 0.80 | 0.77 |
| OAS1 | | | | | | | | | | |
| ROCK2 & | 0.81 | 0.82 | 0.18 | 0.22 | 0.72 | 0.75 | 0.16 | 0.22 | 0.80 | 0.79 |
| TFF3 | | | | | | | | | | |
| DGKA & | 0.75 | 0.77 | 0.20 | 0.26 | 0.71 | 0.76 | 0.17 | 0.23 | 0.80 | 0.81 |
| KIBRA | | | | | | | | | | |
| BRD2 & | 0.88 | 0.88 | 0.15 | 0.17 | 0.70 | 0.73 | 0.16 | 0.21 | 0.76 | 0.75 |
| SYNGR1 | | | | | | | | | | |
| EPS8L2 & | 0.81 | 0.83 | 0.19 | 0.23 | 0.68 | 0.72 | 0.16 | 0.21 | 0.76 | 0.76 |
| PLXNC1 | | | | | | | | | | |
| RIPK5 & | 0.94 | 0.91 | 0.14 | 0.15 | 0.79 | 0.82 | 0.15 | 0.19 | 0.84 | 0.83 |
| ZNF258 | | | | | | | | | | |
| RIPK5 & | 0.81 | 0.83 | 0.18 | 0.21 | 0.81 | 0.83 | 0.15 | 0.18 | 0.88 | 0.86 |
| SPOP | | | | | | | | | | |
| GATAD1 & | 0.94 | 0.94 | 0.11 | 0.12 | 0.79 | 0.81 | 0.14 | 0.18 | 0.84 | 0.83 |
| MTMR1 | | | | | | | | | | |
| RIPK5 & | 0.88 | 0.85 | 0.17 | 0.20 | 0.78 | 0.81 | 0.16 | 0.20 | 0.84 | 0.84 |
| TNFRSF6B | | | | | | | | | | |
| ASCL2 & | 0.88 | 0.88 | 0.15 | 0.17 | 0.74 | 0.76 | 0.16 | 0.21 | 0.80 | 0.77 |
| GATAD1 | | | | | | | | | | |
| LBH & NTS | 1.00 | 0.92 | 0.14 | 0.16 | 0.64 | 0.65 | 0.14 | 0.21 | 0.64 | 0.63 |
| BIRC4 & | 0.88 | 0.87 | 0.15 | 0.18 | 0.70 | 0.74 | 0.16 | 0.22 | 0.76 | 0.75 |
| NTS | | | | | | | | | | |
| DGKA & | 0.88 | 0.88 | 0.15 | 0.16 | 0.78 | 0.80 | 0.16 | 0.19 | 0.84 | 0.83 |
| NTS | | | | | | | | | | |
| Average | 0.87 | 0.86 | 0.16 | 0.19 | 0.73 | 0.76 | 0.15 | 0.20 | 0.79 | 0.79 |

| | Cross- | Cross- | | Cross- | Cross- | Cross- | | |
|---|---|---|---|---|---|---|---|---|
| | validation | validation | Full | validation | validation | validation | | |
| | specificity | specificity | dataset | NPV | NPV | NPV | | |
| Gene pair | s.d. | coeff. var. | NPV | average | s.d. | coeff. var. | Direction | Dirlogp |
| LOXL3 & | 0.14 | 0.17 | 0.95 | 0.94 | 0.09 | 0.09 | pos | 7.99 |
| NTS | | | | | | | | |
| DEPDC4 & | 0.12 | 0.13 | 0.92 | 0.91 | 0.10 | 0.11 | pos | 7.87 |
| NTS | | | | | | | | |
| NTS & | 0.16 | 0.23 | 1.00 | 0.98 | 0.07 | 0.07 | pos | 7.52 |
| PHF14 | | | | | | | | |
| NOTCH2 & | 0.15 | 0.18 | 0.91 | 0.92 | 0.10 | 0.11 | pos | 7.41 |
| RIPK5 | | | | | | | | |
| NTS & | 0.17 | 0.23 | 0.90 | 0.92 | 0.10 | 0.11 | pos | 7.39 |
| ZBTB26 | | | | | | | | |
| RPP30 & | 0.17 | 0.22 | 1.00 | 0.98 | 0.05 | 0.05 | pos | 7.24 |
| SLC24A2 | | | | | | | | |
| NTS & | 0.19 | 0.26 | 0.90 | 0.90 | 0.12 | 0.13 | pos | 7.14 |
| PTPRE | | | | | | | | |
| BMX & | 0.14 | 0.16 | 0.88 | 0.87 | 0.11 | 0.13 | pos | 6.93 |
| DEPDC4 | | | | | | | | |
| PHF14 & | 0.14 | 0.17 | 0.95 | 0.95 | 0.08 | 0.09 | pos | 6.92 |
| PLA2G3 | | | | | | | | |
| NTS & | 0.16 | 0.21 | 0.90 | 0.90 | 0.11 | 0.12 | pos | 6.72 |
| PHLDB2 | | | | | | | | |
| MTBP & | 0.16 | 0.19 | 0.88 | 0.91 | 0.11 | 0.12 | pos | 6.60 |
| SLC24A2 | | | | | | | | |
| FLRT2 & | 0.13 | 0.16 | 0.88 | 0.88 | 0.12 | 0.13 | pos | 6.40 |
| RCSD1 | | | | | | | | |
| MNDA & | 0.20 | 0.37 | 0.87 | 0.88 | 0.15 | 0.17 | pos | 6.20 |
| SCN3B | | | | | | | | |
| MPP7 & | 0.17 | 0.25 | 1.00 | 1.00 | 0.00 | 0.00 | pos | 6.20 |
| SLC24A2 | | | | | | | | |
| CCDC3 & | 0.14 | 0.16 | 0.88 | 0.87 | 0.11 | 0.13 | pos | 6.14 |
| DEPDC4 | | | | | | | | |
| pp9099 & | 0.15 | 0.19 | 0.91 | 0.91 | 0.11 | 0.12 | pos | 6.14 |
| RNF141 | | | | | | | | |
| DEPDC4 & | 0.14 | 0.17 | 0.95 | 0.93 | 0.09 | 0.10 | pos | 6.11 |
| LAPTM4B | | | | | | | | |
| DEPDC4 & | 0.10 | 0.11 | 0.85 | 0.87 | 0.11 | 0.12 | pos | 6.09 |
| PLA2G4A | | | | | | | | |
| LAPTM4B & | 0.17 | 0.23 | 0.95 | 0.92 | 0.11 | 0.12 | pos | 6.05 |
| PLA2G3 | | | | | | | | |
| PLA2G3 & | 0.17 | 0.25 | 0.94 | 0.93 | 0.11 | 0.12 | pos | 5.99 |
| ZNF297B | | | | | | | | |
| GATAD1 & | 0.15 | 0.18 | 0.91 | 0.89 | 0.11 | 0.13 | neg | 5.67 |
| MLX | | | | | | | | |
| ROCK2 & | 0.16 | 0.21 | 0.90 | 0.89 | 0.13 | 0.14 | neg | 5.68 |
| TFF3 | | | | | | | | |
| DGKA & | 0.15 | 0.17 | 0.88 | 0.87 | 0.11 | 0.13 | neg | 5.68 |
| TFF3 | | | | | | | | |
| GOLGA2 & | 0.17 | 0.23 | 0.86 | 0.87 | 0.12 | 0.14 | neg | 5.75 |
| pp9099 | | | | | | | | |
| FBXO33 & | 0.18 | 0.25 | 0.90 | 0.90 | 0.11 | 0.13 | neg | 5.82 |
| RIPK5 | | | | | | | | |
| DGKA & | 0.16 | 0.19 | 0.91 | 0.92 | 0.09 | 0.10 | neg | 5.83 |
| SLAMF6 | | | | | | | | |
| DGKA & | 0.14 | 0.16 | 0.88 | 0.88 | 0.11 | 0.12 | neg | 5.86 |
| TFF3 | | | | | | | | |
| KIBRA & | 0.18 | 0.23 | 0.83 | 0.84 | 0.12 | 0.15 | neg | 5.87 |
| OAS1 | | | | | | | | |
| ROCK2 & | 0.16 | 0.20 | 0.87 | 0.88 | 0.11 | 0.13 | neg | 5.94 |
| TFF3 | | | | | | | | |
| DGKA & | 0.15 | 0.19 | 0.83 | 0.86 | 0.12 | 0.14 | neg | 6.00 |
| KIBRA | | | | | | | | |
| BRD2 & | 0.18 | 0.23 | 0.90 | 0.91 | 0.11 | 0.12 | neg | 6.03 |
| SYNGR1 | | | | | | | | |
| EPS8L2 & | 0.16 | 0.21 | 0.86 | 0.88 | 0.12 | 0.13 | neg | 6.07 |
| PLXNC1 | | | | | | | | |
| RIPK5 & | 0.15 | 0.18 | 0.95 | 0.94 | 0.09 | 0.09 | neg | 6.09 |
| ZNF258 | | | | | | | | |
| RIPK5 & | 0.13 | 0.15 | 0.88 | 0.90 | 0.10 | 0.11 | neg | 6.21 |
| SPOP | | | | | | | | |
| GATAD1 & | 0.15 | 0.18 | 0.95 | 0.96 | 0.07 | 0.08 | neg | 6.31 |
| MTMR1 | | | | | | | | |
| RIPK5 & | 0.15 | 0.18 | 0.91 | 0.91 | 0.10 | 0.11 | neg | 6.40 |
| TNFRSF6B | | | | | | | | |
| ASCL2 & | 0.18 | 0.23 | 0.91 | 0.92 | 0.10 | 0.11 | neg | 6.46 |
| GATAD1 | | | | | | | | |
| LBH & NTS | 0.20 | 0.32 | 1.00 | 0.94 | 0.10 | 0.11 | neg | 6.99 |
| BIRC4 & | 0.18 | 0.24 | 0.90 | 0.91 | 0.11 | 0.12 | neg | 7.49 |
| NTS | | | | | | | | |
| DGKA & | 0.16 | 0.19 | 0.91 | 0.93 | 0.09 | 0.10 | neg | 7.58 |
| NTS | | | | | | | | |
| Average | 0.16 | 0.20 | 0.91 | 0.91 | 0.10 | 0.11 | | |

FIG. 4 illustrates the effect of various levels of zero-mean Gaussian noise numerically added to the measurement data to assess robustness of PIA class separation performance. Added noise is reported as amplitude in multiples of standard deviations of the original measurement values. Sensitivity remained in the 85-90% range in the presence of 1 standard deviation of noise (see FIG. 4a). For specificity, a wider range of values from 65-85% was observed (FIG. 4b). At 2 standard deviations of noise added, a drop to a default level of approximately 55% specificity was observed (see FIG. 4b), compared to a steady, slow decline over many standard deviation levels for sensitivity (see FIG. 4a). At each level of noise added, average sensitivity and average specificity were computed over an ensemble of 5000 independent cross-validation data splits. These results indicate that the PIA class separation performance for the selected gene pairs is very robust, requiring the addition of multiple standard deviations of noise amplitude before the class separation signal is corrupted to the extent that the classes can no longer be separated by PIA.

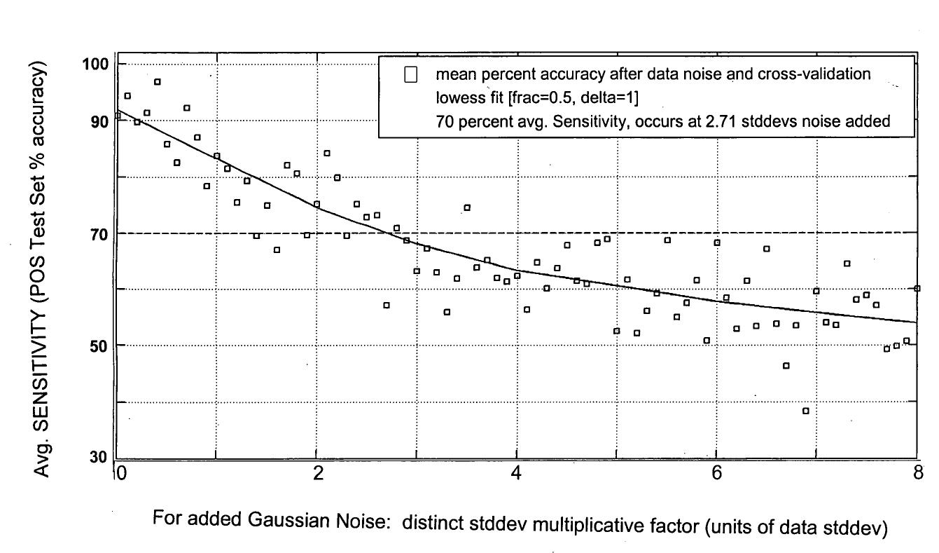

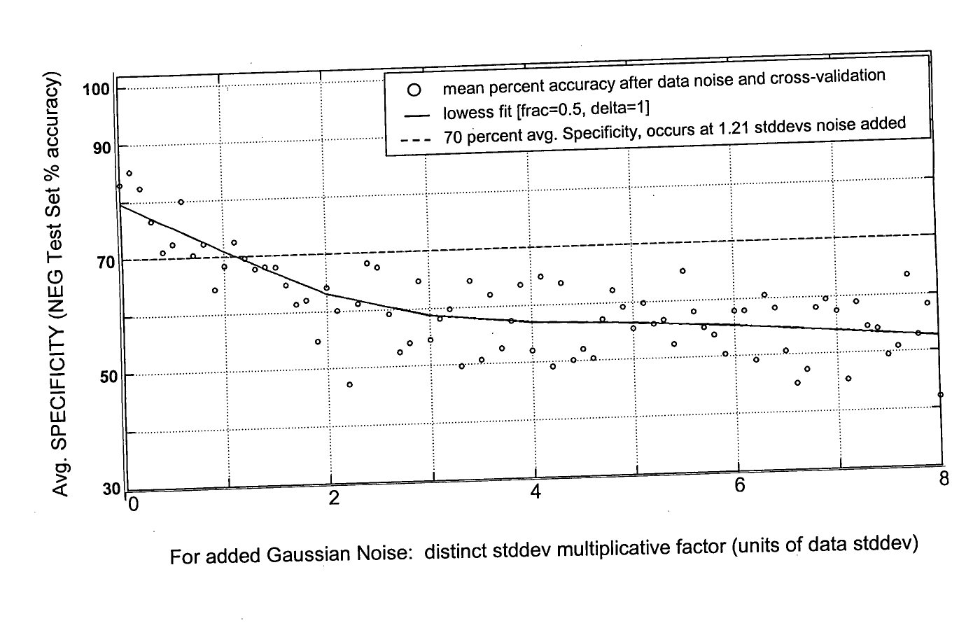

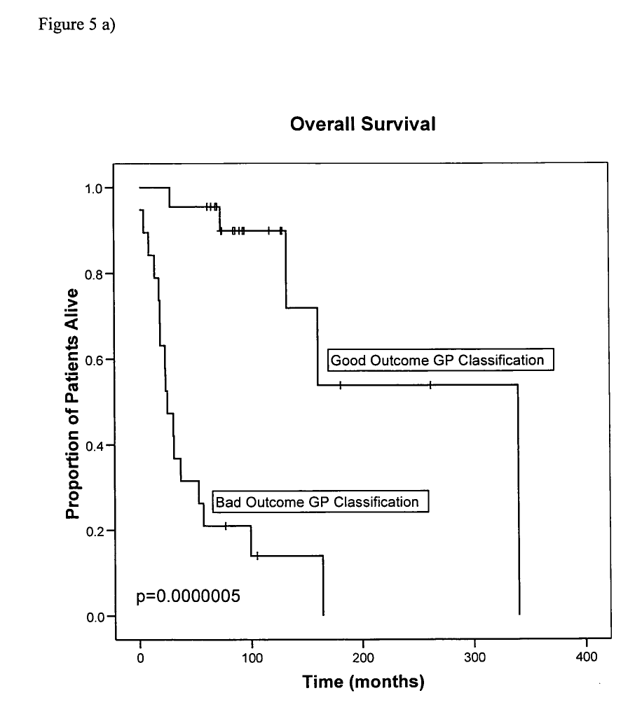

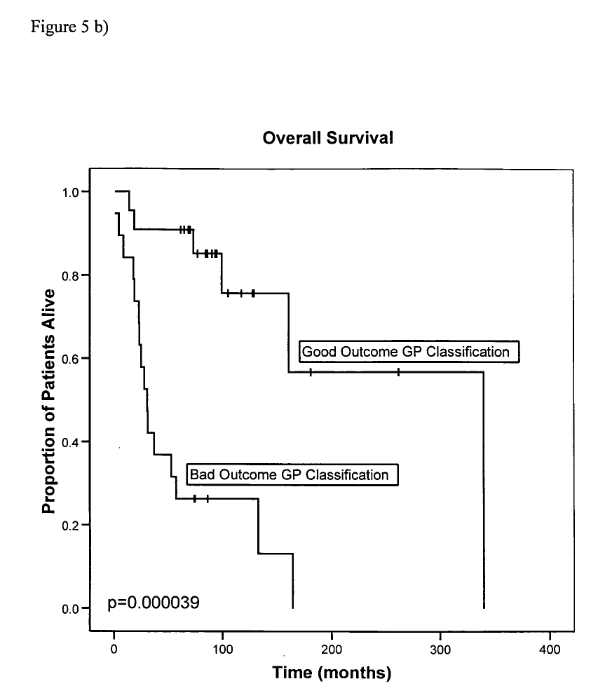

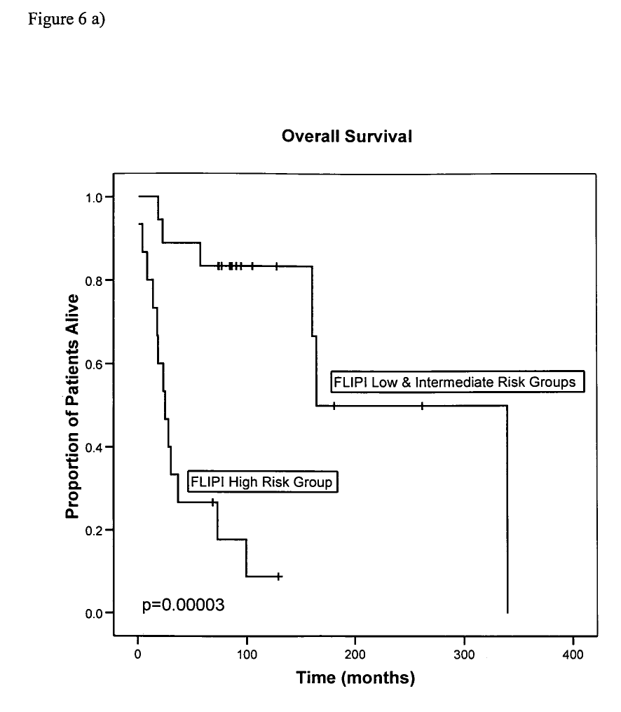

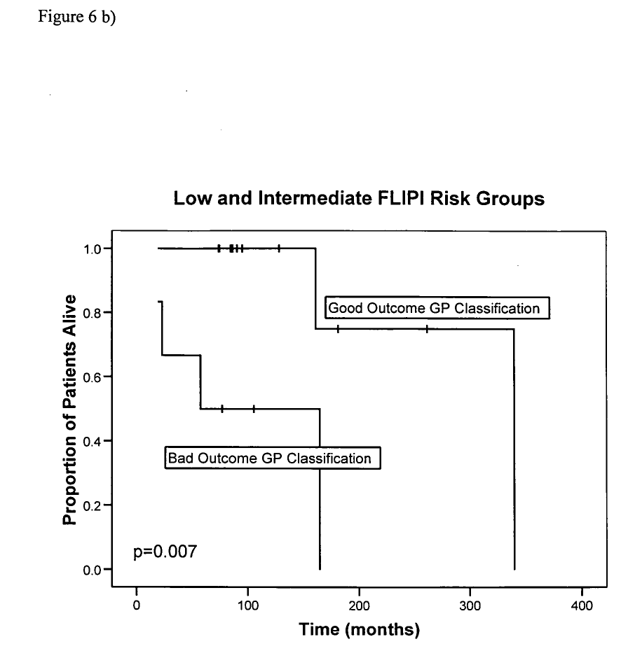

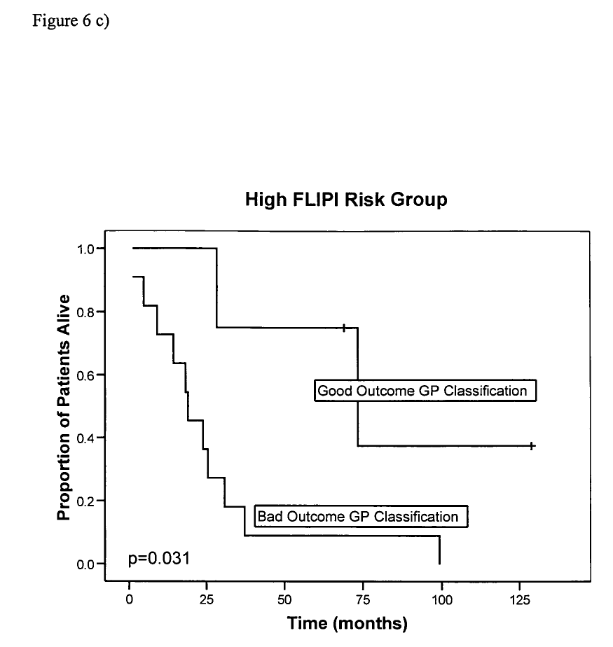

Following identification of the best performing gene pairs, conventional Kaplan-Meier survival analysis curves were generated in order to compare patients' overall survival with segregation based on the combinatorial predictor prognostic classification. The log-rank test was used to assess whether the two groups had significantly different survival curves. As shown in FIG. 5, representative well-performing outcome class-distinguishing gene pairs from PIA were consistently able to divide patients into different prognostic groups when employing Kaplan-Meier survival analysis on the complete survival time information. Patients' FLIPI scores had previously been successfully used to provide prognostic information for FL patients. Due to low numbers of patients in the intermediate risk category, these patients were pooled with low risk patients for analysis purposes. As expected, the FLIPI high risk group had significantly different overall survival than the intermediate and low risk groups combined (see FIG. 6a). As further demonstrated in FIGS. 6b and 6c, stratifying the patients based on FLIPI risk groups, then applying the LOXL3 and NTS gene pair PIA-based patient segregation, for example, was able to further divide the patients in each FLIPI group into significantly different survival curve outcome groups.

The identification of predictive biomarkers for poor outcome in FL described herein differs from prior attempts in a number of important ways. For example, traditional platforms using either long oligo or cDNA probes have required a relative abundance experimental design. In the present invention, however, a one-color, long oligonucleotide microarray platform was used. Use of long oligonucleotide arrays with sufficient internal controls to support one color assays facilitated identification of the predictive biomarkers herein, as this platform removes the confounding issue of a second dye from the data analyses. In addition, inclusion of spike-in RNA controls prior to amplification provided a measure of certainty that the protocols performed as desired, leaving only the question of how well a particular experimental RNA species behaved in that protocol. To that end, the quality control parameters that were measured were indicative of the RNA behaving consistently across samples.

In addition to the quality reports, accuracy of the array data was evident from the data analysis. The presence of multiple genes that had been spotted more than once on the chip provided a means for checking consistency of gene expression measurements for a variety of genes. As shown in FIG. 2a, many of the gene pairs that provided high predictive accuracy were based on genes that were represented by more than one feature on the array, and were identified independently using the same objective analysis criteria.

The approach to data analysis used by the inventors herein was also unique as the analysis did not depend on clustering algorithms, and therefore did not generate large agglomerated sets of genes as classification signatures. Instead, a direct approach of searching for specific gene pair combinations with outcome predictive capabilities that exceeded what the best constituent single genes achieved was taken.

As will be understood by the skilled artisan upon reading this disclosure, alternative methodologies can be used to identify top performing single genes, pairs of which may be predictive of 5 year survival in patients.

For example, 695 top performing single genes were identified from raw values derived from the feature extraction software by first removing all of entries that corresponded to controls, then examining the range of expression values for each gene across all of the slides and removing any genes for which one of the expression values was either less than 5 or greater than 40,000. This effectively trimmed away the very low and the very high expression values. Each slide was then median normalized and the Pearson correlation for each gene with outcome, namely death within five years, was calculated. PIA can be carried out on this dataset as well to identify gene pairs able to discriminate the 5-year outcomes of patients with FL more reliably than single genes of the dataset.

Selected sets of classifier genes were also identified by analysis of a primary dataset consisting of expression data for 41,000 genes from 29 tissue samples of follicular lymphoma classified as having “good” (alive 5 years after diagnosis) or “poor” (dead within three years of diagnosis) outcomes with tumors having a DLBCL component excluded. All analysis was done on the gProcessedSignal data, which had already been subject to extensive normalization by the Agilent software. Analysis was done on log-normalized data.

Two sets of genes, one with 13 and one with 14 genes, were found that classified the data with high accuracy (better than 85% correct for both classes with a p-value of less than 10e-10.) These exemplary sets of classifier genes have been combined and are depicted in Table 3.

TABLE 3
Exemplary gene classifier sets

| Probe Name | Gene Name |
|---|---|
| A_23_P81262 | PCDHB4 |
| A_24_P238365 | ZBTB34 |
| A_24_P221414 | DNCI1^{a} |
| A_32_P80231 | BM973227 |
| A_32_P151454 | AL577308 |
| A_32_P196021 | FGF7 |
| A_32_P3400 | BF754999^{a} |
| A_23_P397455 | ACVR1C |
| A_24_P739344 | NOX4^{a} |
| A_32_P145876 | THC2281706 |
| A_32_P141488 | THC2408967 |
| A_23_P18539 | MMRN1 |
| A_24_P263144 | BMX |
| A_24_P359799 | OTX1 |
| A_24_P68342 | COL4A10 |
| A_24_P36299 | GRLF1 |
| A_23_P431912 | ZNF6452 |
| A_24_P350223 | RaLP |
| A_32_P8653 | A_32_P8653 |
| A_23_P27265 | C18orf4 |
| A_24_P41882 | PDLIM7 |
| A_23_P155185 | ENST00000256031 |
| A_23_P255695 | SLC17A3 |
| A_23_P118158 | HS3ST2 |

^{a}designates gene identified in both exemplary classifier sets.

All analysis was done using an algorithm that finds weighted averages of genes that distinguish classes. The algorithm operates on the log-normalized data and is inherently combinatoric. It can be set to use a wide or narrow acceptance, allowing the resultant gene list size to be varied easily. For example, with wider acceptance criteria gene lists of a few hundred could be found. In the other direction, it may be possible to reduce the number of genes in the classifier gene sets presented here even further. Accordingly, a set of classifier genes of the present invention may comprise more or less genes than listed in Table 3 herein. In one embodiment, the set of classifier genes comprises at least one gene listed in Table 3. In another embodiment, the set of classifier genes comprises two or more genes listed in Table 3. In another embodiment, the set of classifier genes comprises three or more genes listed in Table 3. In another embodiment, the set of classifier genes comprises four or more genes listed in Table 3. In another embodiment, the set of classifier genes comprises five or more genes listed in Table 3. In yet another embodiment, the set of classifier genes comprises ten or more genes listed in Table 3.

A multiple test\training sets computational approach was employed to guard against creating classifiers that can produce high apparent accuracy but have negligible statistical significance. The analysis was carried out in two branches with each branch involving splitting the data into multiple test and training sets.

The “correct-class” branch used the correct classifications for the data. The data were split randomly into approximately equal training and test sets using ten different random partitions. Each training set had 16 samples (9 good, 7 poor) and each test set had 13 samples (7 good, 6 poor). Each test set has no samples in common with its associated training set, so the test set acts as an independent test of any classifier developed by analysis of the training set. Although the training sets have some samples in common, by having ten distinct partitions of the data the effects of using different samples in the analysis can be explored, and measures of how robust the results are can be obtained. In particular, during each iteration of the algorithm a large number of genes are selected as potentially significant. Taking the intersection of the genes found from all 10 training sets substantially reduces this number.

The “random-class” branch of the analysis is identical to the “correct” branch, except in this case the sample class assignments were randomized prior to splitting. This allows determination of the probability of getting the performance seen in the “correct” branch test sets by chance.

The algorithm was run through three iterations, starting with all 41,000 genes. On the first pass it selected approximately 10,000 genes for each correct-class partition, and a similar number for the random-class partitions. The intersection of these gene lists produced an input list for the second pass of 4500 genes in the correct-class branch, and 450 genes in the random-class branch. If each partition was statistically independent of the others, less than 1 gene would be expected to survive the intersection process. The difference between 4500 correct-class genes and 450 random-class genes is consistent with the hypothesis that the correct-class genes are causally related to the classes.

Two more passes reduced the gene lists to between 10 and 15 genes. The analysis was run twice, once with code that took the log of the data on each pass, instead of just at the beginning. This had two effects: negative values were set to a small positive value before taking the log, and positive values were reduced in dynamic range. Because of the particular differences seen in the genes that differentiate between the classes, this approach emphasized the differences between classes. Poor samples had distinguishing genes that were negative or very small values initially, so they had a significantly greater tendency to undergo this non-linear conversion process, resulting in better differentiation in this case. The analysis was then re-run without multiple logs. It produced similar results, and in fact three of the genes in both exemplary classifier gene sets are the same, suggesting a very high level of robustness despite the significant difference between the two cases.

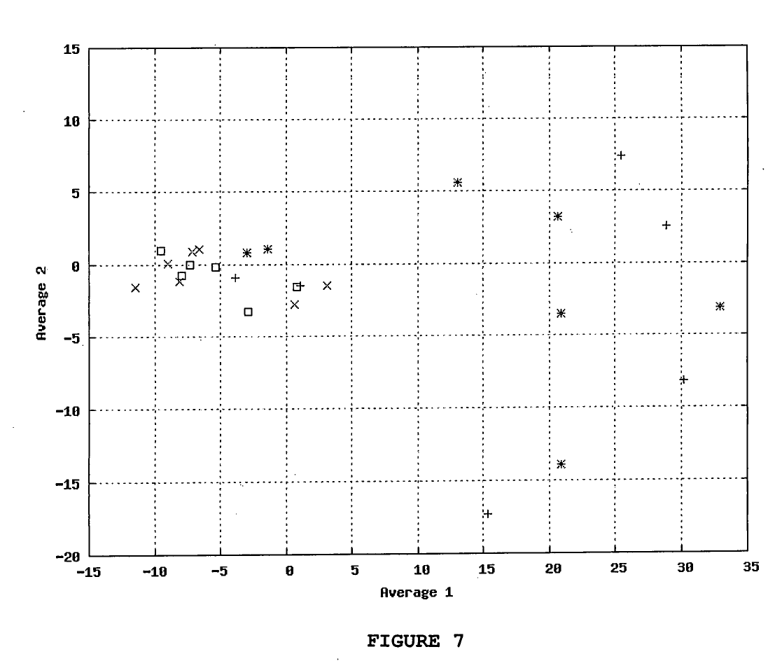

FIG. 7 shows a typical result for a single correct-class partition. As can be seen, almost all the discrimination is done by the first average, which has more-or-less equal, negative, weights. Because the log-normalized good values are typically negative, this gives them positive averages, while the poor value weighted averages are less than zero. The +'s represent samples with good outcome in the training set, the *'s represent samples with good outcomes in the test set. The x's and squares are the same for samples with poor outcomes. The poor outcome samples are strongly clustered between Average 1 values of 5 and −15, while the good outcome samples tend to have much higher values. This is not due to chance.

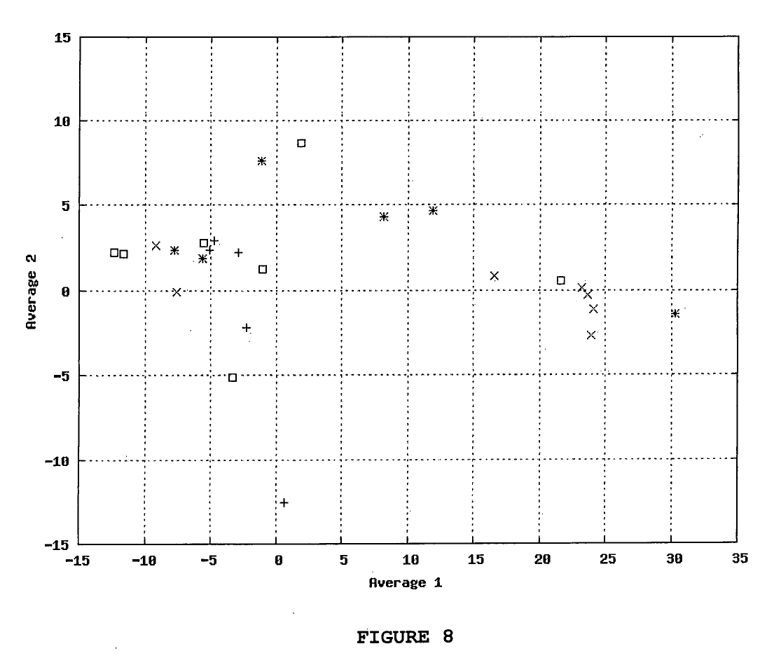

FIG. 8 shows a similar comparison to FIG. 7, but with classes assigned to the samples randomly. In this comparison, moderately good separation of the training data (+'s and x's) is achieved, which is to be expected. However, this separation does not generalize to the test data (*'s and squares). Instead, the squares tend to cluster with the +'s and the *'s with the x's.

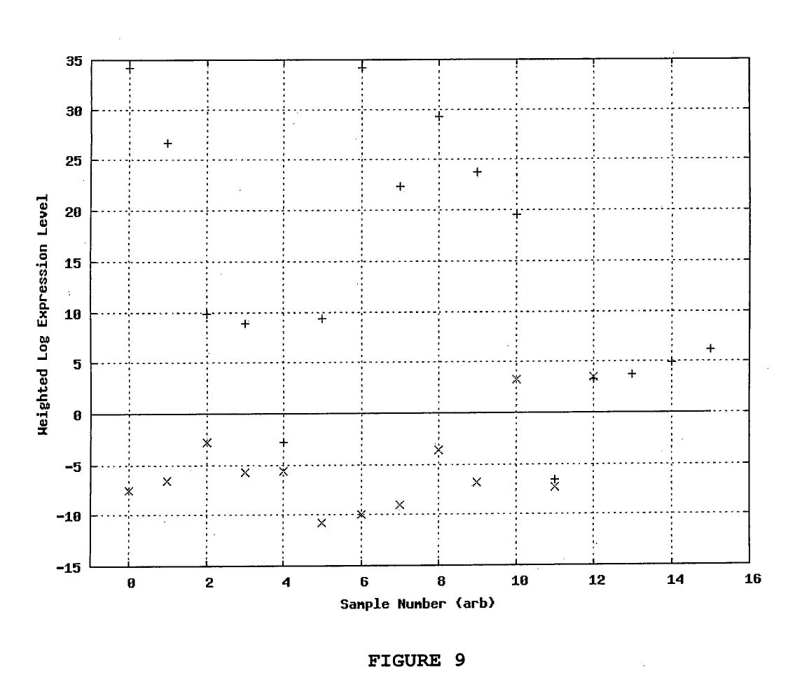

While both averages (FIGS. 7 and 8) have been shown for clarity of presentation, the data can be classified by a simple cut-off value on the first average. This is shown in FIG. 9 for all the data using an averaged classifier. In FIG. 9 the classifier was generated by averaging the weighting values from all the Average 1 classifiers generated from the different partitions of the data. Alternatively, the entire dataset can be run through the analysis process.

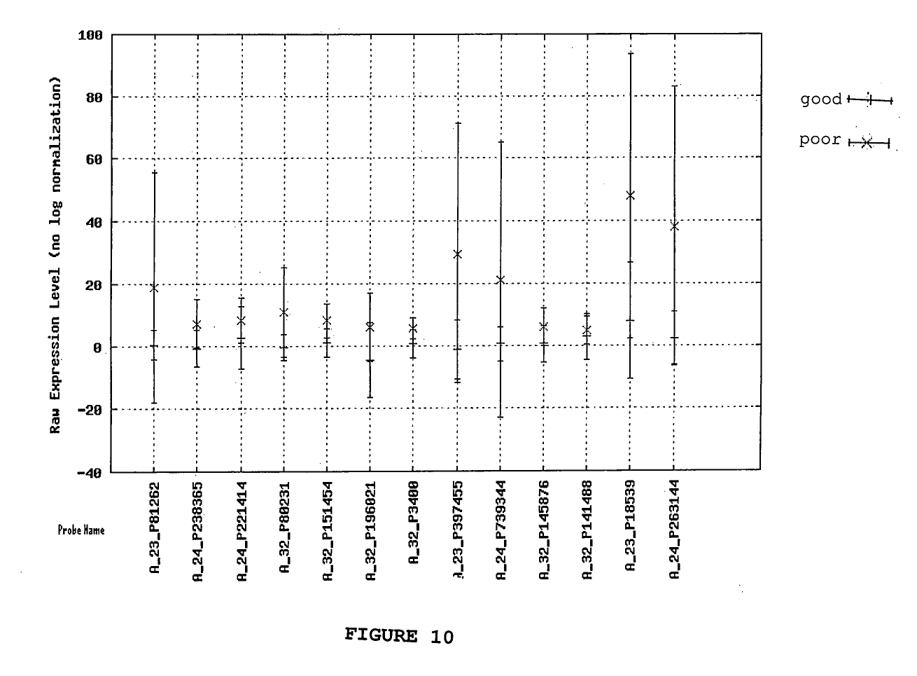

While the weighted-average classifier is quite decisive in distinguishing between good-outcome and poor-outcome samples, distributions of the underlying genes were also examined. These distributions are shown in FIG. 10. While each gene has little statistical significance individually because of the overlap in expression values and the lack of separation by more than one standard deviation, the weighted average using the classifier (see FIG. 9) has a value of 14.2+/−13.0 for the good outcomes and −5.31+/−4.46 for the poor outcomes, indicating a more than one standard deviation separation between the two classes.

Additional methods useful in identifying a set of classifier genes from a list of discriminant genes and thus applicable to the instant invention are described in published U.S. Patent Application No. US2006/0177837.

Also provided in the present invention is a computer system predictive of the outcome of patients with follicular lymphoma. The computer system of the present invention comprises a central processing unit. This system further comprises a memory connected to the central processing unit. The memory can store established levels of expression of one or more gene pairs predictive of 5 year survival or of death within 5 years derived from individuals with follicular lymphoma. Alternatively or in addition, the memory can store multiple gene expression levels of a patient. The system further comprises a computer program capable of comparing levels of expression of one or more gene pairs predictive of 5 year survival or of death within 5 years measured in a patient with stored levels. In one embodiment, the computer program compares levels of expression of one or more gene pairs predictive of 5 year survival or of death within 5 years measured in the patient with stored established levels of expression of one or more gene pairs predictive of 5 year survival or of death within 5 years derived from individuals with follicular lymphoma to predict outcome of the patient. In this embodiment, similar levels of expression in the patient to established levels are indicative of a similar outcome for the patient. In another embodiment, the computer program compares levels of expression of one or more gene pairs predictive of 5 year survival or of death within 5 years measured in the patient with multiple gene expression levels measured in that patient. In this embodiment, a significant increase or decrease in expression of a predictive gene pair or pairs (as set forth above) relative to the multiple gene expression levels is indicative of outcome of the patient. The system further comprises instructions for outputting predicted outcome of the patient based upon the comparison.

The invention is further illustrated by the following examples, which should not be construed as further limiting. The contents of all references, pending patent applications, and published patents cited throughout this application are hereby expressly incorporated by reference.

### EXAMPLES

### Example 1

### Samples and Pathology Review

Cases of FL were identified retrospectively by searching the surgical pathology archive of Kingston General Hospital (Ontario, Canada). The primary criteria for inclusion in the study were: 1) availability of frozen biopsy tissue amenable to the purification of high quality RNA; and, 2) availability of adequate clinical information, including clinical baseline and outcome data based on follow-up for at least 5 years. Forty-one cases were identified in this manner. A portion of biopsy tissue was snap frozen in cryovials containing Tissue Tek Optimal Cutting Temperature compound (Sakura Finetek USA, Inc. Torrance, Calif.) in an isopentane bath shortly after excision and maintained thereafter at −80° C. The routine and immunostained histology slides were retrieved and reviewed by two pathologists in order to confirm the diagnosis of FL and ensure consistent grading according to the World Health Organization criteria.

### Example 2

### Clinical Details

Clinical charts were available for review from all of the patients. Baseline data collected included age at diagnosis, sex, Eastern Cooperative Oncology Group (ECOG) performance status, stage and grade presence of bulky disease, presence of greater than five lymph node areas more than 3 cm in size, number of extranodal sites involved, P2 microglobulin levels, bone marrow involvement, lactic acid dehydrogenase (LDH) levels, hemoglobin, white blood cell count, differential white blood cell count and platelet count. The date of diagnosis, time to transformation, time to death, and time to last follow-up visit were also noted (see Table 1). Treatment modalities and response were noted, as was clinical evidence of tumor progression or transformation to more aggressive disease. Prognostic index scores were calculated using the FLIPI criteria.

### Example 3

### RNA Extraction and Quality Assessment

Total RNA was extracted from each frozen sample using Trizol (Qiagen, Mississauga, Canada) according to manufacturer's recommendation. Each sample was further purified using an RNEasy column clean up (Qiagen). RNA concentration and A₂₆₀/A₂₈₀ ratios were determined using a Nanodrop ND-1000 V-Vis Spectrophotometer (Nanodrop Technologies, Wilmington, Del.), and RNA integrity was measured using a 2100 Bioanalyzer (Agilent, Mississauga, Canada). Based on empirical data from our microarray center, only samples with RNA Integrity Numbers of at least 7 were used for microarray experimentation.

### Example 4

### Microarrays

For each sample, 100 ng of total RNA were mixed with 1 μl of a 5000-fold dilution of Agilent's One Color Spike-in RNA control. The mixture was amplified using the Low Input RNA Amplification kit (Agilent Technologies, Inc., Santa Clara, Calif.). Following amplification and labeling with Cy3, each sample was assessed on the Nanodrop ND-1000 to measure yield and specific activity. Only samples with yields of greater than 1.65 μg cRNA and specific activities greater than 9.0 pmol Cy3/μg cRNA were processed further.

Successfully amplified and labeled samples were hybridized in a rotating oven to Agilent 44K Human Whole Genome microarrays according to manufacturer's instructions. Slides were scanned with an Agilent scanner and quantitated using Agilent Feature Extraction software, Version 8.0.

### Example 5

### Data Analysis

Features with more than 10% missing values across all slides were removed from analysis. All preprocessing and analysis was carried out on the log₁₀ transformed gene expression measurements. Interslide standardization was accomplished using trimmed-mean subtraction across all genes on each slide.

Single genes were analyzed for their ability to predict outcome within 5 years of diagnosis. We carried out Predictive Interaction Analysis (Baron et al. PLOS Med. 2007 4:e23) in accordance with definitions and procedures set forth herein to examine whether any gene pairs from the top 300 single genes thus selected showed statistically enhanced outcome prediction ability.

A model was built on a training data subset and the outcome classification accuracies were established in an independent test set to computationally cross-validate the determinations. Cross-validation was carried out using conventional procedures (Yang YH, S.T. Design and analysis of comparative microarray experiments. In: C. Hall (ed.) Statistical Analysis of Gene Expression Microarray Data, pp. 35-91. Boca Raton:CRC Press, 2003) by randomly selecting 75% of the samples for training (12 positive and 19 negative), and 25% of the samples for testing (4 positive and 6 negative). For each gene pair, 5000 distinct selections of training/test dataset splits were made and all of the four accuracy performance measures were determined.

Conventional Kaplan-Meier survival analysis curves were generated post-hoc using SPSS for Windows version 14.0 (Chicago, Ill.) in order to compare overall survival among the 41 patients. Patient outcome class segregation was based on the best performing gene pairs. For each selected gene pair, patients were classified based on whether their PIA outcome prediction fell within the good outcome or poor outcome group. Overall survival was assessed subsequently by Kaplan-Meier analysis based on patients' survival time according to the PIA predicted outcome class segregation. The conventional log-rank test was used then to assess whether the two groups had significantly different survival curves. Comparisons of patients' overall survival based on FLIPI prognostic groups were made also. Due to low numbers of patients in the FLIPI intermediate risk category, these patients were pooled with low risk patients for analysis purposes. Following stratification of the patient set by FLIPI scores, overall survival using the best performing prognostic gene pairs was assessed.

### Example 6

### Pathway Analysis

Pathway analysis was carried out using the Ingenuity Pathways Analysis (IPA) software tool ((Ingenuity® Systems, Redwood City, Calif., USA)). Briefly, the top 300 gene pairs list was used as input to the program, with no weighting given to predictive strength of any given gene. Pathways were identified from the Ingenuity Pathways Analysis library of canonical pathways that were most represented in the data set. The significance of the association between the data set and the canonical pathway was measured in two ways. First, a ratio of the total number of genes from the dataset that map to the pathway divided by the total number of canonical genes that map to the pathway was provided. Second, a Fisher's exact test was used to calculate a p-value determining the probability that the association between the genes in the dataset and the canonical pathway was explained by chance alone.

What is claimed is:

1. An assay for predicting outcome in a patient with follicular lymphoma comprising means for detection and/or evaluation of expression levels of one or more gene pair(s) or a set of classifier genes predictive of 5 year survival in the patient in a biological sample of the patient.

2. The assay of claim 1 wherein said means detects and/or evaluates expression levels of one or more phenomenologically competitive or synergistic gene pair(s) as identified by Predictive Interaction Analysis.

3. The assay of claim 1 wherein said means detects and/or evaluates expression levels of one or more gene pair(s) of a cell survival pathway.

4. The assay of claim 1 wherein said means detects and/or evaluates expression levels of one or more gene pair(s) involved in apoptosis, chemokine signaling, cell growth and proliferation or hematological function.

5. The assay of claim 1 wherein said means detects and/or evaluates expression levels of one or more gene pair(s) of Table 2.

6. The assay of claim 1 wherein said means detects and/or evaluates expression levels of one or more gene pair(s) selected from the group of genes consisting of NOTCH2, TFF3, CSF1, BNIP1, BCLAF1, BMX, BIRC4, SRF, LIMK1, ROCK2, CCL13, DGKA and TNFRSF6B.

7. The assay of claim 1 wherein expression levels of more than one gene pair are detected and/or evaluated.

8. The assay of claim 1 wherein expression levels of at least one of the genes of the set of classifier genes identified in Table 3 are detected.

9. The assay of claim 1 wherein expression levels of two or more of the genes of the set of classifier genes identified in Table 3 are detected.

10. The assay of claim 1 wherein expression levels of three or more of the genes of the set of classifier genes identified in Table 3 are detected and/or evaluated.

11. The assay of claim 1 wherein expression levels of four or more of the genes of the set of classifier genes identified in Table 3 are detected and/or evaluated.

12. The assay of claim 1 wherein expression levels of five or more of the genes of the set of classifier genes identified in Table 3 are detected.

13. The assay of claim 1 wherein expression levels of at ten or more of the genes of the set of classifier genes identified in Table 3 are detected.

14. A method for prognosticating outcome for a patient with follicular lymphoma, said method comprising detecting and/or evaluating in a biological sample of a patient expression levels of one or more gene pair(s) or a set of classifier genes predictive of 5 year survival in the patient.

15. The method of claim 14 wherein expression levels of one or more phenomenologically competitive or synergistic gene pair(s) as identified by Predictive Interaction Analysis are detected and/or evaluated.

16. The method of claim 14 wherein expression levels of one or more gene pair(s) of a cell survival pathway are detected and/or evaluated.

17. The method of claim 14 wherein expression levels of one or more gene pair(s) involved in apoptosis, chemokine signaling, cell growth and proliferation or hematological function are detected and/or evaluated.

18. The method of claim 14 wherein expression levels of one or more gene pair(s) of Table 2 are detected and/or evaluated.

19. The method of claim 14 wherein expression levels of one or more gene pair(s) selected from the group of genes consisting of NOTCH2, TFF3, CSF1, BNIP1, BCLAF1, BMX, BIRC4, SRF, LIMK1, ROCK2, CCL13, DGKA and TNFRSF6B are detected and/or evaluated.

20. The method of claim 14 wherein expression levels of more than one gene pair are detected and/or evaluated.

21. The method of claim 14 wherein expression levels of at least one of the genes of the set of classifier genes identified in Table 3 are detected.

22. The method of claim 14 wherein expression levels of two or more of the genes of the set of classifier genes identified in Table 3 are detected.

23. The method of claim 14 wherein expression levels of three or more of the genes of the set of classifier genes identified in Table 3 are detected and/or evaluated.

24. The method of claim 14 wherein expression levels of four or more of the genes of the set of classifier genes identified in Table 3 are detected and/or evaluated.

25. The method of claim 14 wherein expression levels of five or more of the genes of the set of classifier genes identified in Table 3 are detected.

26. The method of claim 14 wherein expression levels of at ten or more of the genes of the set of classifier genes identified in Table 3 are detected.

27. A computer system predictive of the outcome of patients with follicular lymphoma, said computer system comprising a central processing unit, a memory connected to the central processing unit, said memory storing established levels of expression of one or more gene pairs or a set of classifier genes predictive of 5 year survival or of death within 5 years derived from individuals with follicular lymphoma and/or multiple gene expression levels of a patient, a computer program capable of comparing levels of expression of said predictive gene pairs or set of classifier genes in the patient with stored levels, and instructions for outputting predicted outcome of the patient.
